(19) United States
(12) Patent Application Publication
(10) Pub. No.: US 2011/0224297 A1
(43) Pub. Date: Sep. 15, 2011

(54) Methods of administering prostratin and structural analogs thereof

(76) Inventors: Stephen J. Brown, West Hollywood, CA (US); Marjan Hezareh, La Jolla, CA (US)

(21) Appl. No.: 12/937,364

(22) PCT Filed: Apr. 13, 2009
(86) PCT No.: PCT/US09/40319

§ 371 Date: May 27, 2011

Related U.S. Application Data
(60) Provisional application No. 61/044,300, filed on Apr. 11, 2008.

(51) Int. Cl. A61K 31/22; A61P 31/18

(52) U.S. Cl. 514/546

(57) ABSTRACT

This invention relates generally to methods for administering prostratin or a structural analog or metabolite thereof to induce latent HIV-1 expression in mammalian cells. In certain embodiments, prostratin or a structural analog or metabolite thereof is administered by infusion. In an exemplary embodiment, the method of administering prostratin or a structural analog or metabolite thereof to induce latent HIV-1 expression further comprises the step of administering HAART. The invention also relates to kits comprising prostratin or a structural analog or metabolite thereof packaged with instructions for infusing the compound to induce latent HIV-1 expression.

### RELATED APPLICATIONS

This application claims priority to U.S. Provisional Patent Application No. 61/044,300, filed Apr. 11, 2008, which is herein incorporated by reference in its entirety.

### FIELD OF THE INVENTION

This invention relates generally to methods for administering prostratin to induce latent HIV-1 expression in mammalian cells.

### BACKGROUND OF THE INVENTION

Antiretroviral drugs have improved the quality of life and decreased the rate of progression to AIDS among HIV positive individuals in developed countries. However, several studies have demonstrated that even in patients with undetectable plasma viremia (<50 copies/ml), virus rebounds after the interruption of Highly Active Anti-Retroviral Therapy (HAART) due to the presence of reservoirs of latently infected cells (Wong et al., 1997, Science 278(5341): 1291-5; Finzi et al, 1997, Science 278(5341): 1295-300; Chun et al., 1997, Nature 387(6629): 183-8). The best-characterized cellular reservoirs for HIV are resting memory CD4+ T-cells. In an HIV-infected individual, some HIV-infected activated CD4+ T-cells may survive both the cell killing effect of the virus and the HIV specific immune responses, and enter a resting state with HIV-1 provirus in their genome (Blankson et al., 2002, Annu Rev Med 53: 557-93; Pierson et al., 2000, Annu Rev Immunol 18: 665-708). Because the transcription of HIV genes depend on the activation state of CD4+ cells, the integrated HIV DNA is transcriptionally silent in these cells, and therefore unaffected by HAART. Once these cells encounter a protein or carbohydrate capable of stimulating an immune response, they become activated and begin to produce virus. The stability of HIV reservoirs is consistent with long-term survival of memory CD4+ cells (over 20 years), the presence of wild-type and drug-resistance HIV strains in reservoirs and the hypothesis that low levels of virus replication is continuously reseeding the reservoirs in patients on HAART (Blankson et al., 2002, Annu Rev Med 53: 557-93; Chun et al., 1997, Nature 387(6629): 183-8; Ruff et al., 2002, J Virol 76(18): 9481-92; Ramratnam et al., 2000, Nature Medicine 6(1): 82-5; Bailey et al., 2006, J Virol 80(13): 6441-57).

A recent study showed that in patients who initiated antiviral therapy early in infection, the reservoir half-life was estimated to be 4.6 months. (Chun et al., 2007, J Infect Dis 195(12): 1762-4). Based upon these findings, Chun and colleagues estimated that it would take up to 7.7 years of continuous therapy to completely eliminate latently infected resting CD4+ T-cells in these individuals. Unfortunately, the long-term use of antiretroviral therapy is associated with side effects including metabolic disorders and cardiovascular disease.

Since the discovery of viral reservoirs, several strategies have been investigated to eliminate HIV reservoirs. One strategy involves the activation of HIV replication in latently infected cells in the continued presence of HAART. The rationale for this strategy is that such cells will die more rapidly due to the cell killing effect of the virus or will present viral components on their surfaces. This in turn will make them more detectable by the immune system and/or render them more susceptible to targeted destruction by immune system cell toxins and other potential therapeutic agents designed to bind selectively to viral products.

Most attempts to activate viral production from latently infected cells have focused on cytokines, lipopolysaccharides, bacterial superantigens, and anti-CD3 antibodies. However, most if not all of these agents are highly toxic and/or have other undesirable side effects. Two approaches have already been explored clinically in HAART-suppressed patients: administration of IL-2 and antibodies to CD3 (OKT3). While these strategies are potentially promising, their in-vivo application remains limited by the fact that treatment with IL-2 or anti-CD3 causes the non-specific activation of a large number of T-cells and therefore significant toxicity.

Because of the problems associated with currently known HIV-activating agents, there is an urgent need to investigate the effects of new compounds on the elimination of viral reservoirs. Recent studies have shown that prostratin may be an important potential candidate for further development in new anti-HIV therapeutic protocols because of its ability to induce latent HIV-1 expression in viral reservoirs.

Prostratin, a 12-deoxyphorbol ester and an activator of protein kinase C (PKC), was initially isolated at the National Cancer Institute (NCl) as the active constituent of extracts of the tropical plant, Homalanthus nutans, which was used in traditional Samoan herbal medicine for treatment of “yellow fever,” i.e., hepatitis (Gustafson et al., 1992, J Med Chem 35(11): 1978-86). In contrast to most other phorbol esters, prostratin is not a tumor-promoter but is actually a potent anti-tumor agent. Thus, prostratin represents a distinct subclass of PKC activators, which differs in its biological activities from tumor-promoting phorbol esters such as PMA.

Studies have shown that prostratin is a potent activator of HIV expression in latently infected cells. Prostratin up-regulates expression of viral products from latently infected cells such as U1, ACH-2 cell lines and resting CD4+ T-cells (Kulkosky et al., 2001, Blood 98(10): 3006-15; Gustafson et al., 1992, J Med Chem 35(11): 1978-86; Gulakowski et al., 1997, Antiviral Res 33(2): 87-97; Biancotto et al., 2004, J Virol 78(19): 10507-15). Korin and colleagues demonstrated that prostratin alone was able to activate latent HIV expression in thymocytes and PBMCs from SCID-hu mice, with similar potency as anti-CD3 and anti-CD28 co-stimulation (Korin et al., 2002, J Virol 76(16): 8118-23). Further investigation on the effect of prostratin demonstrated that prostratin, either alone or in conjunction with other activators, stimulates HIV-1 production from PBMCs in 4 out of 6 individuals on suppressive HAART (Kulkosky et al., 2001, Blood 98(10): 3006-15). Studies in SCID-hu mice demonstrated that prostratin in combination with immunotoxin can effectively and specifically eliminate HIV viral reservoirs (Brooks et al., 2003, Immunity 19(3): 413-23).

Against this background, the present inventors aimed to develop methods for effectively and safely administering prostratin as an adjunct to HAART for the elimination of latent viral reservoirs.

### SUMMARY OF THE INVENTION

The present inventors have found that administration of prostratin via infusion maintains the concentration of drug at levels sufficient to activate latent viral reservoirs, while at the same time, being low enough to avoid the potentially harmful side effects associated with prostratin therapy.

The results of several experiments by the present inventors clearly demonstrate that prostratin is not a tumor promoter as opposed to other phorbol esters and should be administered at a low dosage as an infusion to keep the concentration of drug stable over a period where it can activate viral reservoirs.

The present invention provides methods and kits that provide for the administration of prostratin to induce latent HIV-1 expression in mammalian cells.

In one aspect, the invention provides a method for inducing latent HIV-1 expression in a mammalian cell, the method comprising administering to a mammal in need thereof a dosage amount of about 2.5 μg/kg/hr to about 50 μg/kg/hr of prostratin or a structural analog thereof by infusion for about 2 hours to about 72 hours. In certain embodiments, prostratin is administered by infusion for about 4 hours to about 24 hours. In an exemplary embodiment, prostratin is administered at a concentration of about 5 μg/kg/hr to about 15 μg/kg/hr by infusion for about 6 hours. In another exemplary embodiment, the mammalian cell is in a human.

In one embodiment of the invention, administration of prostratin or a structural analog thereof by infusion is performed using an infusion pump. In certain embodiments, prostratin or a structural analog thereof is administered via infusion using intravenous, intraarterial, intralymphatic, or intraperitoneal administration.

In another embodiment of the invention, administration of prostratin or a structural analog thereof is performed in combination with the administration of HAART. This method, known as a Reservoir Ablative Strategy (RAS), has potential applications for eliminating latent HIV reservoirs.

In another aspect, the invention provides a kit for inducing latent HIV-1 expression in a mammalian cell, comprising prostratin or a structural analog thereof packaged with instructions for infusing the compound to induce latent HIV-1 expression. In certain embodiments, the kit further comprises a pharmaceutically acceptable carrier, excipient, or diluent. In an exemplary embodiment, the kit includes prostratin or a structural analog thereof in a form suitable for intravenous infusion.

In yet another aspect, the invention provides a method for administering prostratin or a structural analog or a prodrug thereof as an orally active sustained release formulation. In one embodiment, the sustained release formulation is administered orally in tablet form at least once, twice, or three times over a 24 hour period. In certain embodiments, the effective plasma concentration attained by the sustained release formulation is sustained for at least 4 hours. In an exemplary embodiment, the effective plasma concentration attained by the sustained release formulation is between about 50 ng/ml and about 150 ng/ml.

### BRIEF DESCRIPTION OF THE DRAWINGS

FIG. 1. Representative chromatogram of prostratin and an apparent human microsome-derived metabolite shown at 7.6 min.

FIG. 2. Effects of prostratin stimulating HIV expression in latently infected ACH-2 and U1 cells in the continuous presence of prostratin. Continuous incubation means that prostratin or PMA was left in the medium for time of cell incubation at 37° C. following stimulation.

FIG. 3. Effects of prostratin stimulating HIV expression after a short period of incubation in ACH-2 cells. Short incubation means that cells were stimulated with prostratin for the indicated times (30 min, 1, 4, and 6 hours), then washed and incubated with fresh medium without prostratin for the rest of the incubation at 37° C.

FIG. 4. Effects of prostratin on HIV expression after a short period of incubation in U1 cells.

FIG. 5. Pharmacokinetics of prostratin after an i.v. (A) and i.p. (B) injection in mice.

FIG. 6. Levels of AST and ALT in monkeys after i.v. bolus injection of prostratin.

FIG. 7. Levels of CK and LD after i.v. bolus injection of prostratin in monkeys.

FIG. 8. IL-6 level in monkeys after i.v. bolus injection of prostratin.

FIG. 9. Plasma concentration of prostratin in monkeys.

FIG. 10. Concentration of prostratin in plasma sample of monkeys.

### DETAILED DESCRIPTION

The present invention relates generally to methods and kits that enable the administration of prostratin or a structural analog thereof by infusion to induce latent HIV-1 expression in mammalian cells.

The method of the invention comprises administering the phorbol ester, prostratin (12-deoxyphorbol 13-acetate), or a structural analog thereof, or a metabolite thereof, and a pharmaceutically acceptable carrier, diluent, or excipient.

As used herein, the term “pharmaceutically acceptable” refers to those properties and/or substances which are acceptable to the patient from a pharmacological/toxicological point of view and to the manufacturing pharmaceutical chemist from a physical/chemical point of view regarding factors such as formulation, stability, patient acceptance and bioavailability. Suitable carriers for use in the present invention include, but are not limited to, injectable or orally administerable oils, lipid emulsions or aqueous suspensions, or in the case of orally administerable tablets or capsules, a pharmacologically inert excipient.

Prostratin and structural analogs thereof may be purified from a natural source or may be synthetically made. Methods for synthetically producing prostratin and structural analogs are known in the art (Wender et al., 2008, Science 320(5876): 649-52).

In certain embodiments, structural analogs of prostratin may be used to induce latent HIV-1 expression. As used herein, the term “structural analog” means a compound that shares structural characteristics with prostratin, but differ structurally in other ways, such as the inclusion or deletion of one or more other chemical moieties. For example, a structural analog of prostratin may share one or more structural characteristics with the parent prostratin compound, such as the 12-deoxyphorbol 13-monoester structure, but may differ in which ester is selected. The ester may be selected from the group consisting of formate, acetate, propionate, butyrate, pentanoate, hexanoate, benzoate, and phenylacetate. In an exemplary embodiment, the ester is acetate (12-deoxyphorbol 13-acetate (prostratin)). In other embodiments, the ester may be, for example, phenylacetate (12-deoxyphorbol 13-phenylacetate (DPP)). The present inventors have shown that the phorbol ester DPP displays similar effects to prostratin with regard to activation of HIV latency.

In certain embodiments, structural analogs or derivatives of prostratin may be used to induce latent HIV-1 expression. In one embodiment, the prostratin derivative is a 12-deoxyphorbol derivative. By “12-deoxyphorbol derivative”, it is meant a structural analog of phorbol which does not have an oxygen atom attached to position 12 of the core structure. Certain 12-deoxyphorbol derivatives are described in WO 2007/009055, the content of which is herein incorporated by reference in its entirety. In one embodiment, the 12-deoxyphorbol derivative is a 12-deoxyphorbol ester derivative. In another embodiment, the structural analog of prostratin is a compound of formula (I):

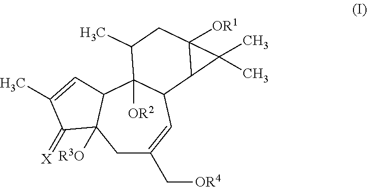

or a pharmaceutically acceptable salt or solvate thereof;

wherein,

R¹, R², R³, and R⁴ are each independently —O(CO)OR⁵, —O(CO)N(R⁵)₂, —O(CO)R⁶, or a structural formula selected from the group consisting of

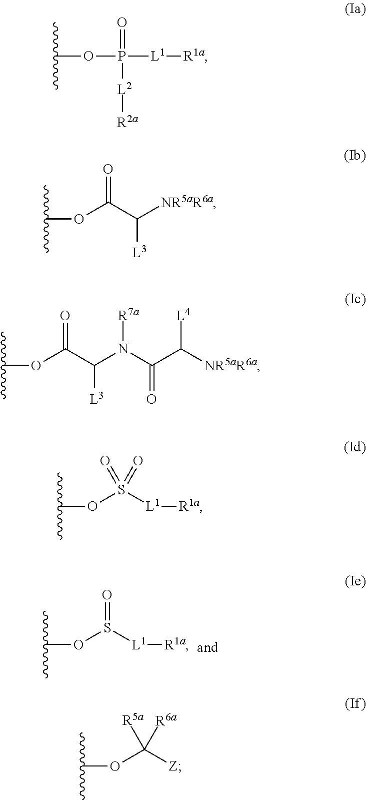

L¹ and L² are each independently a covalent bond, —O—, or —NR^{3a}—;

R^{1a} and R^{2a} are each independently hydrogen, alkyl, heteroalkyl, heteroaryl, heterocyclyl, alkenyl, alkynyl, arylalkyl, heteroarylalkyl, heterocyclylalkyl, -alkylene-C(O)—O—R^{4a}, or -alkylene-O—C(O)—O—R^{4a}; and

R^{3a} and R^{4a} are each independently hydrogen, alkyl, heteroalkyl, cyclylalkyl, heterocyclyl, aryl, heteroaryl, alkenyl, alkynyl, arylalkyl, heterocyclylalkyl, or heteroarylalkyl;

L³ and L⁴ are each independently hydrogen, halogen, nitro, cyano, alkyl, alkenyl, alkynyl, arylalkyl, aryl, heteroalkyl, heterocyclyl, heteroaryl, heterocyclylalkyl, heteroarylalkyl, OR5, N(R⁵)₂, or SR⁵;

R^{5a}, R^{6a}, and R^{7a} are each independently hydrogen, alkyl, alkenyl, alkynyl, alkylaryl, arylalkyl, aryl, heteroalkyl, alkylheteroaryl, heterocyclyl, or heteroaryl;

Z has a structural formula selected from the group consisting of (Ia), (Ib), (Ic), (Id), and (Ie);

X is O, S, or NR⁵;

each R⁵ is independently hydrogen, alkyl, substituted alkyl, alkenyl, substituted alkenyl, aryl, substituted aryl, heteroaryl, or substituted heteroaryl; and

R⁶ is alkyl, substituted alkyl, alkenyl, substituted alkenyl, aryl, substituted aryl, heteroaryl, or substituted heteroaryl.

In one embodiment of formula (I), the compound is not prostratin.

In one embodiment of formula (I), X is oxygen.

In one embodiment of formula (I), one of R² and R³ is hydrogen. In another embodiment, R² and R³ are both hydrogen.

In one embodiment of formula (I), the compound has a structural formula (II):

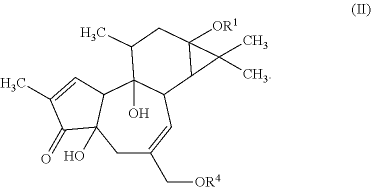

In one embodiment of formula (II), R⁴ is hydrogen.

In one embodiment of formula (II), R¹ is —O(CO)R⁶; and R⁴ is hydrogen. In other words, in one embodiment of formula (II), the compound is a 12-deoxyphorbol 13-monoester. The ester may be selected from the group consisting of formate, acetate, propionate, butyrate, pentanoate, hexanoate, benzoate, and phenylacetate. In an exemplary embodiment, the ester is acetate (12-deoxyphorbol 13-acetate (prostratin)). In other embodiments, the ester may be, for example, phenylacetate (12-deoxyphorbol 13-phenylacetate (DPP)). The present inventors have shown that the phorbol ester DPP displays similar effects to prostratin with regard to activation of HIV latency.

In certain embodiments, the structural analogs of prostratin include prodrugs of prostratin. As used herein, the term “prodrug” is intended to include derivatives of prostratin, which after administration undergo conversion to prostratin. The prodrug includes a compound that is metabolized, for example hydrolyzed or oxidized, in the host to form prostratin. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. In certain embodiments, prodrugs of prostratin may be used to induce latent HIV-1 expression.

The terms “prostratin”, “structural analog of prostratin”, and “prostratin derivative” refer to compounds encompassed by structural formulae disclosed herein and includes any specific compounds within these formulae. Prostratin or prostratin derivative described herein contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. In one embodiment, the compound of formula (I) has the following stereochemistry:

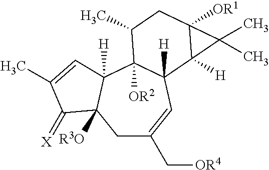

Prostratin or prostratin derivative described herein may also exist in several tautomeric forms including the enol form, the keto form and mixtures thereof. Accordingly, the chemical structures depicted herein encompass all possible tautomeric forms of the illustrated compounds. The term “tautomer” as used herein refers to isomers that change into one another with great ease so that they can exist together in equilibrium.

Prostratin or prostratin derivative described herein also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴C, ¹⁵N, ¹⁸O, ¹⁷O, etc.

Prostratin or prostratin derivatives described herein may exist in unsolvated forms as well as solvated forms, including hydrated forms and as N-oxides. In general, compounds may be hydrated, solvated or N-oxides.

Prostratin or prostratin derivatives described herein may exist in multiple crystalline or amorphous forms. In general, all physical forms are equivalent for the uses contemplated herein and are intended to be within the scope of the present invention.

Whenever a term in the specification is identified as a range (i.e. C₁₋₄ alkyl), the range independently refers to each element of the range. As a non-limiting example, C₁₋₄ alkyl means, independently, C₁, C₂, C₃ or C₄ alkyl. Similarly, when one or more substituents are referred to as being “independently selected from” a group, this means that each substituent can be any element of that group, and any combination of these groups can be separated from the group. For example, if R¹ and R² can be independently selected from X, Y and Z, this separately includes the groups R¹ is X and R² is X; R¹ is X and R² is Y; R¹ is X and R² is Z; R¹ is Y and R² is X; R¹ is Y and R² is Y; R¹ is Y and R² is Z; R¹ is Z and R² is X; R¹ is Z and R² is Y; and R¹ is Z and R² is Z.

The term “alkyl”, by themselves or as part of other substituents, refers to a saturated straight, branched, or cyclic, primary, secondary, or tertiary hydrocarbon, including but not limited to groups with C₁ to C₁₀. The term “alkyl” includes “lower alkyl”. The term “lower alkyl” refers to a saturated straight, branched, or cyclic, primary, secondary, or tertiary hydrocarbon, including groups with C₁ to C₄, and if appropriate a cyclic alkyl group (for example cyclopropyl). The term “alkyl” also includes “cycloalkyl”, “heteroalkyl”, “heterocycloalkyl”, “arylalkyl”, and “heterarylalkyl” as defined herein below.

Illustrative examples of alkyl groups are methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless otherwise specified, the alkyl group can be unsubstituted or substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991

The term “halo” or “halogen”, as used herein, includes chloro, bromo, iodo, and fluoro.

The term “chiral” as used herein includes a compound that has the property that it is not superimposable on its mirror image.

The term “alkylthio” refers to a straight or branched chain alkylsulfide of the number of carbons specified, such as for example, C₁₋₄alkylthio, ethylthio, —S-alkyl, —S-alkenyl, —S-alkynyl, etc.

The terms “alkylamino” or “arylamino” refer to an amino group that has one or two alkyl or aryl substituents, respectively. Unless otherwise specifically stated in this application, when alkyl is a suitable moiety, then it is a lower alkyl, whether substituted or unsubstituted.

The term “alkylsulfonyl” means a straight or branched alkylsulfone of the number of carbon atoms specified, as for example, C₁₋₆ alkylsulfonyl or methylsulfonyl.

The term “alkoxycarbonyl” refers to a straight or branched chain ester of a carboxylic acid derivative of the number of carbon atoms specified, such as for example, a methoxycarbonyl, MeOCO—.

As used herein, the term “nitro” means NO₂; the term “sulfhydryl” means —SH; and the term “sulfonyl” means —SO₂.

The terms “alkenyl” and “alkynyl”, by themselves or as part of other substituents, refer to alkyl moieties, including both substituted and unsubstituted forms wherein at least one saturated C—C bond is replaced by a double or triple bond. Thus, C₂₋₆ alkenyl may be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl. Similarly, C₂₋₆ alkynyl may be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

The term “alkylene” includes a saturated, straight chain, divalent alkyl radical of the formula —(CH₂)ₙ—, wherein “n” may be any whole integer from 1 to 10.

“Alkyl”, “alkoxy”, “alkenyl”, “alkynyl”, etc., includes both straight chain and branched groups. However, reference to an individual radical such as “propyl” embraces only that straight-chain radical, whereas a branched chain isomer such as “isopropyl” is specifically termed such.

The term “aryl”, by themselves or as part of other substituents, as used herein and unless otherwise specified refers to any stable monocyclic, bicyclic, or tricyclic carbon ring of up to 8 members in each ring, wherein at least one ring is aromatic as defined by the Huckel 4n+2 rule, and especially phenyl, biphenyl, or naphthyl. The term includes both substituted and unsubstituted moieties. The aryl group can be substituted with any described moiety, including but not limited to one or more moieties selected from the group consisting of halogen (fluoro, chloro, bromo or iodo), hydroxyl, amino, azido, alkylamino, arylamino, alkoxy, aryloxy, nitro, cyano, sulfonic acid, sulfate, phosphonic acid, phosphate, or phosphonate, either protected or unprotected as necessary, as known to those skilled in the art, for example, as taught in Greene et al., Protective Groups in Organic Synthesis, John Wiley & Sons, 3^{rd} Ed., 1999.

The term “alkaryl” or “alkylaryl” refers to an alkyl group with an aryl substituent or an alkyl group linked to the molecule through an aryl group as defined herein. The term “aralkyl” or “arylalkyl” refers to an aryl group substituted with an alkyl substituent or linked to the molecule through an alkyl group as defined above.

The term “cycloalkyl”, by themselves or as part of other substituents, includes a ring of C₃₋₈, including but not limited to cyclopropyl, cyclobutyl, cyclopentyl, cyclohexyl, cycloheptyl and cyclooctyl.

The term “alkoxy” means a straight or branched chain alkyl group having an attached oxygen radical, the alkyl group having the number of carbons specified or any number within this range. For example, a “−O-alkyl”, C₁₋₄ alkoxy, methoxy, etc.

The term “acyl” or “O-linked ester” includes a group of the formula C(O)R′, wherein R′ is an straight, branched, or cyclic alkyl (including lower alkyl), carboxylate residue of an amino acid, aryl including phenyl, heteroaryl, alkaryl, aralkyl including benzyl, alkoxyalkyl including methoxymethyl, aryloxyalkyl such as phenoxymethyl; or substituted alkyl (including lower alkyl), aryl including phenyl optionally substituted with chloro, bromo, fluoro, iodo, C₁ to C₄ alkyl or C₁ to C₄ alkoxy, sulfonate esters such as alkyl or aralkyl sulphonyl including methanesulfonyl, the mono, di or triphosphate ester, trityl or monomethoxy-trityl, substituted benzyl, alkaryl, aralkyl including benzyl, alkoxyalkyl including methoxymethyl, aryloxyalkyl such as phenoxymethyl. Aryl groups in the esters optimally comprise a phenyl group. In nonlimiting embodiments, acyl groups include acetyl, trifluoroacetyl, methylacetyl, cyclopropylacetyl, cyclopropyl-carboxy, propionyl, butyryl, isobutyryl, hexanoyl, heptanoyloctanoyl, neo-heptanoyl, phenylacetyl, 2-acetoxy-2-phenylacetyl, diphenylacetyl, α-methoxy-α-trifluoromethyl-phenylacetyl, bromoacetyl, 2-nitro-benzeneacetyl, 4-chloro-benzeneacetyl, 2-chloro-2,2-diphenylacetyl, 2-chloro-2-phenylacetyl, trimethylacetyl, chlorodifluoroacetyl, perfluoroacetyl, fluoroacetyl, bromodifluoroacetyl, methoxyacetyl, 2-thiopheneacetyl, chlorosulfonylacetyl, 3-methoxyphenylacetyl, phenoxyacetyl, tert-butylacetyl, trichloroacetyl, monochloro-acetyl, dichloroacetyl, 7H-dodecafluoro-heptanoyl, perfluoro-heptanoyl, 7H-dodeca-fluoroheptanoyl, 7-chlorododecafluoro-heptanoyl, 7-chloro-dodecafluoro-heptanoyl, 7H-dodecafluoroheptanoyl, 7H-dodeca-fluoroheptanoyl, nona-fluoro-3,6-dioxa-heptanoyl, nonafluoro-3,6-dioxaheptanoyl, perfluoroheptanoyl, methoxybenzoyl, methyl 3-amino-5-phenylthiophene-2-carboxyl, 3,6-dichloro-2-methoxy-benzoyl, 4-(1,1,2,2-tetrafluoro-ethoxy)-benzoyl, 2-bromo-propionyl, omega-aminocapryl, decanoyl, n-pentadecanoyl, stearyl, 3-cyclopentyl-propionyl, 1-benzene-carboxyl, O-acetylmandelyl, pivaloyl acetyl, 1-adamantane-carboxyl, cyclohexane-carboxyl, 2,6-pyridinedicarboxyl, cyclopropane-carboxyl, cyclobutane-carboxyl, perfluorocyclohexyl carboxyl, 4-methylbenzoyl, chloromethyl isoxazolyl carbonyl, perfluorocyclohexyl carboxyl, crotonyl, 1-methyl-1H-indazole-3-carbonyl, 2-propenyl, isovaleryl, 1-pyrrolidinecarbonyl, 4-phenylbenzoyl.

The term “acylamino” includes a group having a structure of “—N(R′)—C(═O)—R′”, wherein each R′ is independently as defined above.

The term “carbonyl” includes a group of the structure “—C(═O)—X—R′” or “X—C(═O)—R′”, where X is O, S, or a bond, and each R is independently as defined above.

The term “heteroatom” includes an atom other than carbon or hydrogen in the structure of a heterocyclic compound, nonlimiting examples of which are nitrogen, oxygen, sulfur, phosphorus or boron.

The term “heteroalkyl”, by themselves or as part of other substituents, refer to an alkyl group in which one or more of the carbon atoms (and optionally any associated hydrogen atoms), are each, independently of one another, replaced with the same or different heteroatoms or heteroatomic groups. The heteroatoms or heteroatomic groups may be placed at any interior position of the alkyl group.

The term “cycloheteroalkyl” by itself or as part of another substituent, refers to a cyclic alkyl radical in which one or more carbon atoms (and optionally any associated hydrogen atoms) are independently replaced with the same or different heteroatom.

The term “heterocycle”, “heterocyclyl”, or “heterocyclic” as used herein includes non-aromatic ring systems having four to fourteen members, preferably five to ten, in which one or more ring carbons, preferably one to four, are each replaced by a heteroatom. Heterocycle includes, but is not limited to, cycloheteroalkyl. Examples of heterocyclic rings include 3-1H-benzimidazol-2-one, (1-substituted)-2-oxo-benzimidazol-3-yl, 2-tetrahydro-furanyl, 3-tetrahydrofuranyl, 2-tetrahydropyranyl, 3-tetrahydropyranyl, 4-tetra-hydropyranyl, [1,3]-dioxalanyl, [1,3]-dithiolanyl, [1,3]-dioxanyl, 2-tetra-hydro-thiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

The term “heteroaryl”, used alone or as part of a larger moiety as in “heteroaralkyl” or “heteroarylalkoxy”, refers to heteroaromatic ring groups having five to fourteen members. Examples of heteroaryl rings include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, and benzoisoxazolyl. Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquino-linyl, and pyrido[3,4-d]pyrimidinyl. The term “heteroaryl” also refers to rings that are optionally substituted. The term “heteroaryl” may be used interchangeably with the term “heteroaryl ring” or the term “heteroaromatic”.

The term “amino” as used herein unless otherwise specified, includes a moiety represented by the structure “—NR₂”, and includes primary, secondary and tertiary amines optionally substituted by alkyl, aryl, heterocyclyl, and/or sulfonyl groups. Thus R₂ may represent two hydrogen atoms, two alkyl moieties, or one hydrogen and one alkyl moiety.

The term “amido” as used herein includes an amino-substituted carbonyl, while the term “amidino” means a group having the structure “—C(═NH)—NH₂”.

The term “quaternary amine” as used herein includes quaternary ammonium salts that have a positively charged nitrogen. They are formed by the reaction between a basic nitrogen in the compound of interest and an appropriate quaternizing agent such as, for example, methyliodide or benzyliodide. Appropriate counterions accompanying a quaternary amine include acetate, trifluoroacetate, chloro, bromo and iodo ions.

It should be understood that the above-mentioned functional groups, such as alkyl, alkenyl, alkynyl, heteroalkyl, aryl, heteroaryl, arylalkyl, alkylaryl, etc, include the substituted form of those functional groups, i.e., substituted alkyl, substituted alkenyl, substituted alkynyl, substituted heteroalkyl, substituted aryl, substituted heteroaryl, substituted arylalkyl, substituted alkylaryl, etc., The term “substituted” includes multiple degrees of substitution by one or more named substituents such as, for example, halo, hydroxyl, thio, alkyl, alkenyl, alkynyl, nitro, cyano, azido, amino, carboxamido, etc. Where multiple substituent possibilities exist, the compound can be substituted by one or more of the disclosed or claimed substituent groups, independently from one another, and taken singly or plurally.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term “protecting group” as used herein refers to a group that may be attached to a reactive group, including heteroatoms such as oxygen or nitrogen, to prevent the reactive group from participating in a reaction. Any protecting groups taught in Greene, et al., Protective Groups in Organic Synthesis, John Wiley and Sons, Second Edition, 1991 may be used. Examples of suitable protecting groups include but are not limited to alkoxyalkyl groups such as ethoxymethyl and methoxymethyl; silyl protecting groups, such tert-butyldimethyl silyl (TBS), phenyldimethylsilyl, trimethylsilyl (TMS), 2-trimethylsilylethoxymethyl (SEM) and 2-trimethylsilylethyl; and benzyl and substituted benzyl.

It should be understood that the various possible stereoisomers of the groups mentioned above and herein are within the meaning of the individual terms and examples, unless otherwise specified. As an illustrative example, “1-methyl-butyl” exists in both (R) and the (S) form, thus, both (R)-1-methyl-butyl and (S)-1-methyl-butyl is covered by the term “1-methyl-butyl”, unless otherwise specified.

“Salt thereof” means any acid and/or base addition salt of a compound of the present invention. The term “salt thereof” includes but is not limited to pharmaceutically acceptable salt thereof.

“Solvate thereof” means a compound of the present invention formed by solvation (the combination of solvent molecules with molecules or ions of the solute), or an aggregate that consists of a solute ion or molecule with one or more solvent molecules. One example of solvent is hydrate. The term “solvate thereof” includes but is not limited to pharmaceutically acceptable solvate thereof.

“Ester thereof” means any ester of a compound of the present invention in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The term “ester thereof” includes but is not limited to pharmaceutically acceptable ester thereof.

“Pharmaceutically acceptable” means a salt, solvate, and/or ester of a compound of the present invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use.

Where applicable and compatible with the chemical properties of the compound of the present invention, “pharmaceutically acceptable salt” includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

In cases where compounds are sufficiently basic or acidic to form stable nontoxic acid or base salts, administration of the compounds as salts may be appropriate. The term pharmaceutically acceptable salts or complexes refers to salts or complexes that retain the desired biological activity of the compounds of the present invention and exhibit minimal undesired toxicological effects.

Nonlimiting examples of such salts are (a) acid addition salts formed with inorganic acids such as sulfate, nitrate, bicarbonate, and carbonate salts (for example, hydrochloric acid, hydrobromic acid, sulfuric acid, phosphoric acid, nitric acid, and the like), and salts formed with organic acids including tosylate, methanesulfonate, acetate, citrate, malonate, tartarate, succinate, benzoate, ascorbate, α-ketoglutarate, and α-glycerophosphate salts, such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, lithium and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art.

Pharmaceutically acceptable salts may be obtained using standard procedures well known in the art, for example by reacting a sufficiently basic compound such as an amine with a suitable acid affording a physiologically acceptable anion.

Prostratin or prostratin derivative described herein can be prepared via synthetic methods and procedures generally known to one skilled in the art. In certain embodiments, prostratin or prostratin derivative described herein are prepared from phorbol which can be readily isolated from croton oil. For example, prostratin or prostratin derivative described herein can be prepared according to the method and procedure described by Wender et al., “Practical Synthesis of Prostratin, DPP, and Their Analogs, Adjuvant Leads Against Latent HIV”, Science, May 2008, Vol. 320, No. 5876, pages 649-652, the content of which is herein incorporated by reference in its entirety.

In certain embodiments, prostratin or structural analogs thereof can be formulated for parenteral administration by injection, for example, by bolus injection or infusion. Formulations for injection can be presented in unit dosage form, for example, in ampoules or in multi-dose containers, with an added preservative. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. Alternatively, prostratin or structural analogs thereof can be in powder form for constitution with a suitable vehicle before use.

In general, prostratin or structural analogs thereof as active agents are prepared in a pharmaceutically acceptable composition for delivery to a host. The terms “active agent,” “drug,” “agent,” “therapeutic agent,” and the like are used interchangeably herein. Pharmaceutically acceptable carriers preferred for use with a subject agent may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, and microparticles, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. A composition comprising a subject agent may also be lyophilized using means well known in the art, for subsequent reconstitution and use according to the invention.

Prostratin or structural analogs thereof can be administered to an individual in need thereof in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In pharmaceutical dosage forms, prostratin or structural analogs thereof may be administered in the form of its pharmaceutically acceptable salts, or it may also be used alone or in appropriate association, as well as in combination, with other pharmaceutically active compounds. The methods and excipients disclosed herein are merely exemplary and are in no way limiting.

For oral preparations, prostratin or structural analogs thereof can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Prostratin or structural analogs thereof can be formulated into preparations for injection by dissolving, suspending or emulsifying it in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for oral administration such as syrups, elixirs, and suspensions may be provided wherein each dosage unit, for example, teaspoonful, tablespoonful, or tablet contains a predetermined amount of the composition containing one or more active agents. Similarly, unit dosage forms for injection or intravenous administration may comprise prostratin or structural analogs thereof in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of prostratin, calculated in an amount sufficient to produce the desired effect in association with a pharmaceutically acceptable diluent, carrier or vehicle. As is understand by one of ordinary skill in the art, the specifications for a given active agent will depend in part on the particular compound employed and the effect to be achieved, and the pharmacodynamics associated with each compound in the host.

In an exemplary embodiment, the invention provides a method for administering to a mammal in need thereof prostratin or a structural analog thereof by infusion. As used herein, the term “mammal in need thereof” refers to any mammal, including, but not limited to murines, felines, simians, humans, and mammalian livestock in need of treatment for a condition or disease. An exemplary condition is a condition affected or caused by a latent HIV infection.

In some embodiments of the invention, administration of prostratin or a structural analog thereof occurs via infusion using an infusion pump. As used herein, the term “infusion” refers to the generally continuous, slow introduction of fluid into the body, and especially into a vein. As used herein, the term “infusion” refers generally to intravenous infusions, intraarterial infusions, intralymphatic infusions, or intraperitoneal infusions, and like methods of providing a substantially continuous drug dosage over time, so as to maintain an effective serum concentration of the compound within a defined range over time.

As used herein, the term “infusion pump” generally refers to a low infusion rate pressurizing device used in administering or infusing medical fluids. Infusion pumps are designed to provide controlled dosages of medication to a subject, and are utilized to administer fluids in ways that would otherwise be impractically expensive or unreliable if performed manually by hospital staff. For example, infusion pumps can administer as little as 0.1 mL per hour doses (too small for a drip), doses every minute, or doses with repeated pulses of infusion as instructed by a physician. Continuous or substantially continuous infusion usually consists of small pulses of infusion, usually between about 20 nl and about 100 μL, depending on the pump's design, with the rate of these pulses depending on the programmed infusion speed.

In other embodiments of the invention, the pharmaceutical compositions containing prostratin or a structural analog thereof are administered orally. The oral dosage forms may be in the form of tablets, troches, lozenges, aqueous, solid or semi-solid solutions or mixtures, or oily suspensions or solutions, dispersible powders or granules, emulsions, multiparticulate formulations, syrups, elixirs, and the like.

In certain exemplary embodiments, the oral dosage form is a sustained release carrier that effectuates the sustained release of prostratin or a structural analog thereof when the dosage form contacts gastrointestinal fluid. The sustained release dosage form may comprise a multiplicity of substrates and carriers that include prostratin or a structural analog thereof. The substrates may comprise matrix spheroids or may comprise inert pharmaceutically acceptable beads that are coated with prostratin or a structural analog thereof. The coated beads are then preferably overcoated with a sustained release coating comprising the sustained release carrier. The matrix spheroid may include the sustained release carrier in the matrix itself, or the matrix may comprise a simple disintegrating or prompt release matrix containing prostratin or a structural analog thereof, the matrix having a coating applied thereon which comprises the sustained release carrier. In yet other embodiments, the oral solid dosage form comprises a tablet core containing prostratin or a structural analog thereof within a normal or prompt release matrix with the tablet core being coated with a sustained release coating comprising the sustained release carrier.

The method of the invention comprises administration of prostratin or a structural analogs thereof to induce latent HIV-1 expression. As used herein, the term “induce” means the activation of an integrated latent HIV-1 provirus to begin gene expression, eventually leading to the production of infectious virus particles.

As used herein, the term “latent” or “latency” refers to the integration of a HIV-1 provirus within the host cell genome and is characterized by the absence of non-spliced HIV-1 RNA or virus production (Biancotto et al., 2004, J Virol 78(19): 10507-15).

The methods of the present invention can be applied to any cell in which an HIV genome is integrated into the cellular DNA. In an exemplary embodiment, the HIV genome is integrated into the genome of a human cell. In some embodiments, the cell infected is a resting lymphoid mononuclear cell, for example, lymphocytes, such as T cells (CD4, CD8, cytolytic, helper), natural killer cells, and B cells. In an exemplary embodiment, the resting lymphoid mononuclear cell is a CD4+ T cell.

The methods of the present invention may also be practiced further comprising the step of administering HAART sequentially or simultaneously. HAART therapies are often combinations or “cocktails” of two or more antiretroviral agents. HAART includes reverse transcriptase inhibitors and protease inhibitors. Drugs used in HAART regimens include the nucleoside analogs AZT, stavudine (d4T), and 3TC; nevirapine (a non-nucleoside reverse transcriptase inhibitor, which may be abbreviated NVP), and protease inhibitors such as RTV, SQV, IDV, and nelfinavir. Although HAART reduces the viral load in many patients to levels below the current limits of detection, the rapid mutation rate of this virus limits the efficacy of this therapy (Perrin et al., 1998, Science 280: 1871-3). Moreover, HAART is ineffective in treating latent HIV.

Suitable human dosages for these HAART compounds can vary widely. However, such dosages can readily be determined by those of skill in the art. Therapeutically effective amounts of these drugs are administered during HAART. By “therapeutically effective amount” is intended an amount of the antiretroviral agent that is sufficient to decrease the effects of HIV infection, or an amount that is sufficient to favorably influence the pharmacokinetic profile of one or more of the other antiretroviral agents used in the HAART protocol. By “favorably influence” is intended that the antiretroviral agent, when administered in a therapeutically effective amount, affects the metabolism of one or more of the other antiretroviral agents used in HAART, such that the bioavailability of the other agent or other agents is increased. This can allow for decreased dosage frequency of the antiretroviral agent or agents whose bioavailability is increased in this manner Decrease in dosage frequency can be advantageous for antiretroviral agents having undesirable side effects when administered in the absence of the antiretroviral agent that increases their bioavailability. The therapeutically effective dose of an antiretroviral agent for purposes of having a favorable influence on the pharmacokinetics of another antiretroviral agent used in the HAART protocol is typically lower than the amount to be administered to have a direct therapeutic effect on HIV, such as inhibition of HIV replication. When used in this manner, an antiretroviral agent that has undesirable adverse effects at the full dosage required for therapeutic effectiveness against HIV replication can provide a therapeutic benefit a lower doses with fewer adverse side affects.

Thus, in one embodiment, an antiretroviral agent, when administered in a therapeutically effective amount to an HIV-infected subject, decreases the effects of HIV infection by, for example, inhibiting replication of HIV, thereby decreasing viral load in the subject undergoing therapy using the reservoir ablative strategy. In another embodiment, an antiretroviral agent, when administered in a therapeutically effective amount to an HIV-infected subject, favorably influences the pharmacokinetics of one or more of the other antiretroviral agents used in the HAART.

For example, the protease inhibitor ritonavir when administered at full doses is a potent inhibitor of HIV in serum and lymph nodes. When administered for these purposes, adverse reactions are common, such as gastrointestinal intolerance, hyperglycemia, insulin resistance, new onset or worsening diabetes, increased bleeding in hemophiliacs, circumoral and peripheral paresthesias, altered taste, and nausea and vomiting. Ritonavir can be administered at low doses (for example, 100 to 400 mg bid) with minimal intrinsic antiviral activity to increase the serum concentrations and decrease the dosage frequency of other protease inhibitors (see, Hsu et al. (1998) Clin. Pharmacokinet. 35:275). See, for example, the favorable influence of ritonavir on the protease inhibitor lopinavir (ABT-378) (Eron et al. (1999) ICAAC 39 addendum: 18, Abstract LB-20).

Guidance as to dosages for any given antiretroviral agent is available in the art and includes administering commercially available agents at their recommended dosages. See, for example, Medical Letter 42 (Jan. 10, 2000): 16. Thus, for example, IDV can be administered at a dosage of about 800 mg, three times a day; D4T can be administered at a dosage of about 30-40 mg, twice a day; and Nelfinavir can be administered at a dosage of about 1250 mg, twice a day, or 750 mg three times a day. These agents are generally administered in oral formulations, though any suitable means of administration known in the art may be utilized for their delivery.

The present invention also provides kits for inducing latent HIV-1 expression in a mammalian cell. Kits with unit doses of prostratin or structural analogs thereof, e.g. in oral or injectable doses suitable for infusion (e.g., for intramuscular, intravenous, or intralymphatic infusion), are provided. In such kits, in addition to the containers containing the unit doses will be an informational package insert describing the use and attendant benefits of the drugs in treating a latent HIV infection. Suitable active agents and unit doses are those described herein above.

In some embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, formulation containers, and the like.

In certain embodiments, a subject kit includes one or more components or features that increase patient compliance, e.g., a component or system to aid the patient in remembering to take the active agent at the appropriate time or interval. Such components include, but are not limited to, a calendaring system to aid the patient in remembering to take the active agent at the appropriate time or interval.

In some embodiments, prostratin or structural analogs thereof are packaged for oral administration. The present invention provides a packaging unit comprising daily dosage units of prostratin or structural analogs thereof. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

Additional objects, advantages, and novel features of the present invention will become apparent to one of ordinary skill in the art upon examination of the following examples, which are not intended to be limiting. Additionally, each of the various embodiments and aspects of the present invention delineated hereinabove and as claimed in the claims section below finds experimental support in the following examples.

### Examples

### Example 1

### Development and Validation of a Bio-Analytical (LC/MS/MS) Method for Prostratin in Mouse, Rat, Non-Human Primate and Human Plasma

A bioanalytic (LC/MS/MS) assay for prostratin was developed. Briefly, the plasma extraction procedure is as follows: a 100 μl plasma standard was added to 1.5 ml microcentrifuge tube, 100 μl 95:5 methanol:1 nitric acid (vol/vol) was added, the mix was vortexed for 10-15 sec and incubated in ice a minimum of 20 minutes. Following centrifugation at 15000 rpm for 10 min at 5° C. in an Eppendorf 5403 table top centrifuge, 125 ml of clear supernatant was transferred into 200 μl polypropylene HPLC vials for analysis by LC/MS/MS.

Samples (25 μl) are injected onto Water Xterra MS C18 (3.5 λm; 2×10 mm) analytical column connected to an agilent Series 1100 HPLC equipped with autoinjector. The HPLC, in turn, is connected to a microMass Quatro Ultima MS/MS instrument that is run in an electrospray positive mode. The mass transition 413.2>353.1 is used to identify and quantitate the compound. Neat as well as extracted (plasma) solutions of prostratin produce a linear response down to a lower limit of 0.5 ng/ml. Determination of prostratin is best achieved if a curve from 0.5 to 100 ng/ml and a second curve from 10 to 1000 ng/ml are used.

Control mouse plasma samples (n=3) were spiked with a solution of “neat” prostratin and then extracted as indicated above. Percent relative deviation of calculated concentration from nominal concentration and the percent relative error, a measure of accuracy, were calculated and are shown in Table 1.

The data indicate assay reproducibility, accuracy, and precision are within acceptable limits. The assay has also been validated for prostratin in monkey, rat, and human plasma. Assay validation results were similar to those shown above for mouse plasma.

TABLE 1

| | | | | |
|---|---|---|---|---|
| | | Calculated Conc. | Percent | Percent |
| | Nominal Conc. | (ng/ml) | Relative | Relative Error |
| Matrix | (ng/ml) | Mean ± SD | Deviation | (Accuracy) |

LC/MS/MS assay validation for prostratin.

| | | | | |
|---|---|---|---|---|
| Mouse | 0.5 | 0.52 ± 0.05 | 9.61 | 4.00 |
| Plasma | 1 | 1.06 ± 0.05 | 5.09 | 6.0 |
| | 5 | 5.15 ± 0.48 | 9.32 | 3.00 |
| | 10 | 9.15 ± 0.02 | 0.22 | −8.5 |
| | 50 | 54.17 ± 1.84 | 3.39 | 8.34 |
| | 100 | 96.03 ± 3.27 | 3.41 | −3.96 |
| | 500 | 500.77 ± 13.85 | 2.77 | 0.15 |

Between Day reproducibility and accuracy:

| | | | | |
|---|---|---|---|---|
| Day 1 | 10 | 9.15 ± 0.02 | 0.22 | −8.5 |
| Day 2 | 10 | 9.24 ± 0.27 | 2.92 | −7.6 |
| Day 3 | 10 | 10.93 ± 1.21 | 11.07 | 9.3 |
| Day 1 | 100 | 96.03 ± 3.27 | 3.41 | −3.96 |
| Day 2 | 100 | 94.58 ± 5.46 | 5.77 | −5.42 |
| Day 3 | 100 | 97.59 ± 4.99 | 5.11 | −2.41 |

### Example 2

### Stability and Protein Binding

Prostratin is stable in plasma and buffer as well as in the stability formulation (see below) developed for in vivo studies in mice. Stability tests for prostratin in mouse and monkey plasma were done using two concentration (1 and 10 mg/ml) and two temperatures (Room Temperature (RT) and 37° C.). The only loss of drug occurred in mouse plasma after 24 hr incubation at 37° C. This represented only 14% decrease in parent drug peak area over this time frame. Prostratin is stable in PBS (phosphate buffer saline) at pH 2 and pH 7.

Drug was dissolved in vehicle (10% DMSO: 10% Cremophor: 80%:0.9% Saline) and then incubated at four different temperatures (37° C., RT, 4° C., and −20° C.) for up to 2 hr. Prostratin is stable in this formulation even at 37° C. for at least 2 hr.

### Example 3

### In Vitro Metabolism in Mouse, Rat, Monkey and Human Liver Microsomes

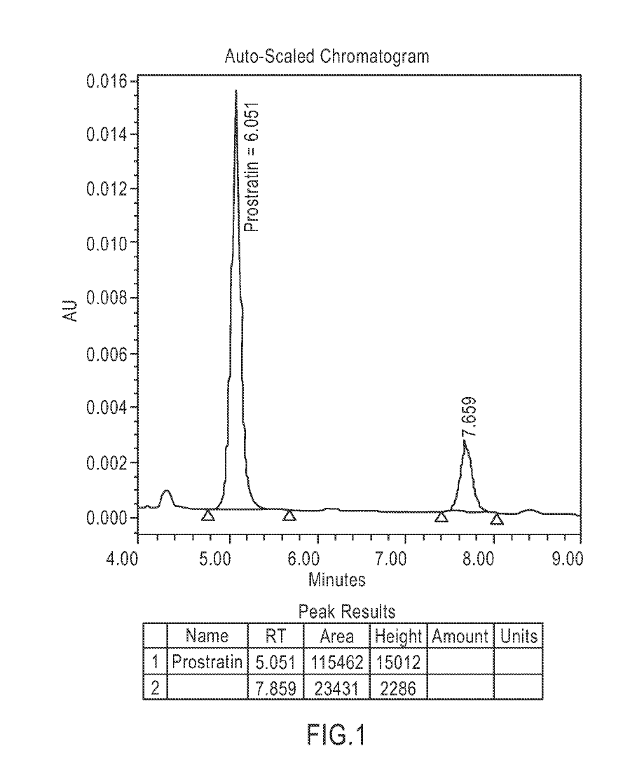

In vitro metabolism of prostratin was investigated using mouse, rat, monkey, and human liver microsomes (Gentest). Time-dependent loss of parent compound and the appearance of a single, later eluting metabolite were observed with each species. Metabolism was greatest with mouse and monkey microsomes and substantially less with rat and human microsomes. However, appearance of metabolite was maximal at 1 hr (over 4 hr incubation time) and the rate of formation did not closely match the loss of parent compound, suggesting that other processes may be involved. Attempts to identify the metabolite by LC/MS/MS were unsuccessful due to the low amounts present in the reaction mixtures (FIG. 1).

### Example 4

### Historic Concentration of Administered Prostratin from Samoa

Prostratin was an active constituent of Homalanthus Nutans, used in Western Samoa to treat a variety of viral diseases such as hepatitis. In Samoa, healers prepare a tea from the bark of the mamala tree. Two samples of this tea, prepared by two different healers, were brought back from Western Samoa. Samples were stored at −70° C. Patients consume about 250 ml of the tea per treatment. Sample was analyzed to determine the concentration of prostratin in undiluted samples and then to calculate the concentration of prostratin/dose. Analyses of these two samples indicated that tea concentration was between 74-145.7 μg/dose.

### Example 5

### Time- and Dose-Response Activity of Prostratin to Stimulate HIV Expression in Latently-Infected ACH-2 and U1 Cells

The lowest concentration of prostratin in an in vitro time-dose exposure was 0.1 μm for an exposure of 24 to 48 hours. Since this concentration can only be achieved transiently in vivo because of toxicity, the choice was made to investigate effective doses of prostratin in latently infected cells for shorter periods of time.

The effect of prostratin on virus replication was assessed in latently infected ACH-2 and U1 cells by measuring the accumulation of reverse transcriptase activity in the supernatant of stimulated cells (Gustafson et al., 1992, J Med Chem 35(11): 1978-86).

Briefly, 4×10⁴ cells in 100 μl fresh medium were mixed with another 100 μl of medium with or without prostratin or PMA in a 96-well microtiter plate. Plates were incubated for 24-72 h at 37° C. Following incubation, supernatants were harvested, centrifuged (to remove any residual cells) and tested for reverse transcriptase activity as previously described (Gustafson et al., 1992, J Med Chem 35(11): 1978-86).

In these experiments, two concentrations of cells were tested (40,000 and 100,000 cells/well). It is noteworthy that the basal levels of reverse transcriptase activities were much higher in ACH-2 cells than in U1 cells.

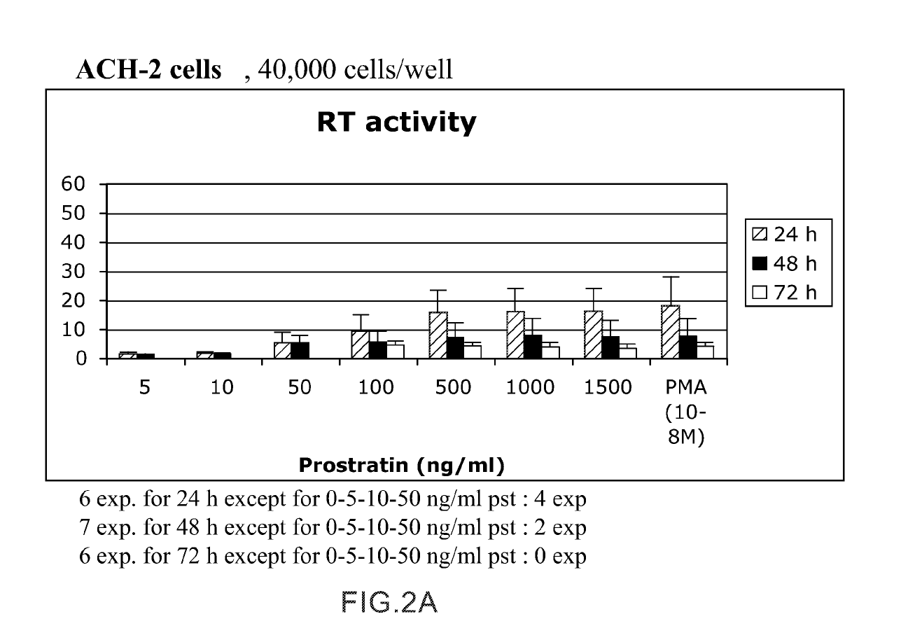

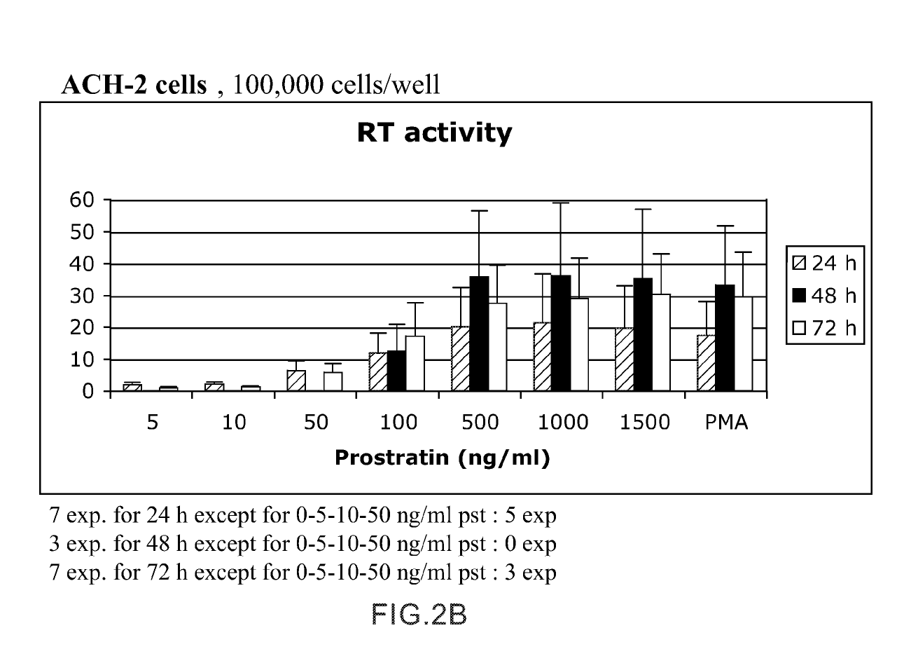

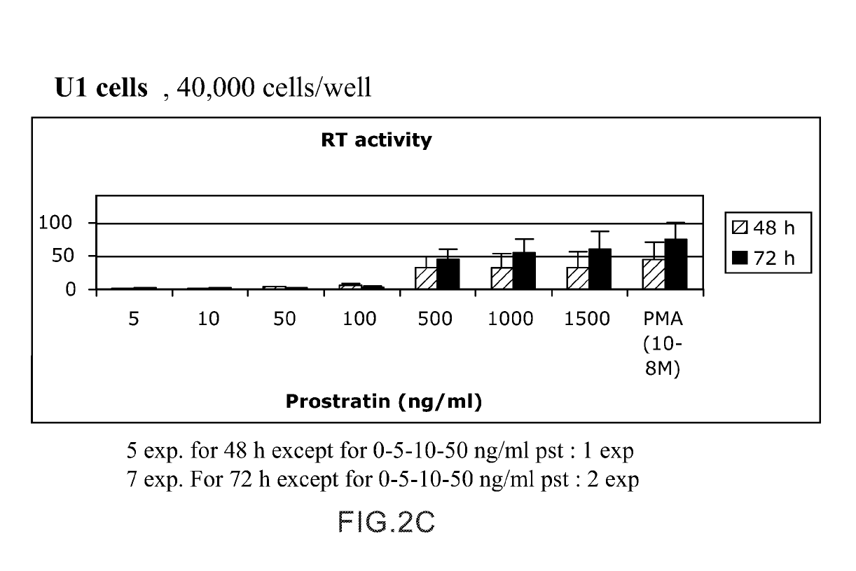

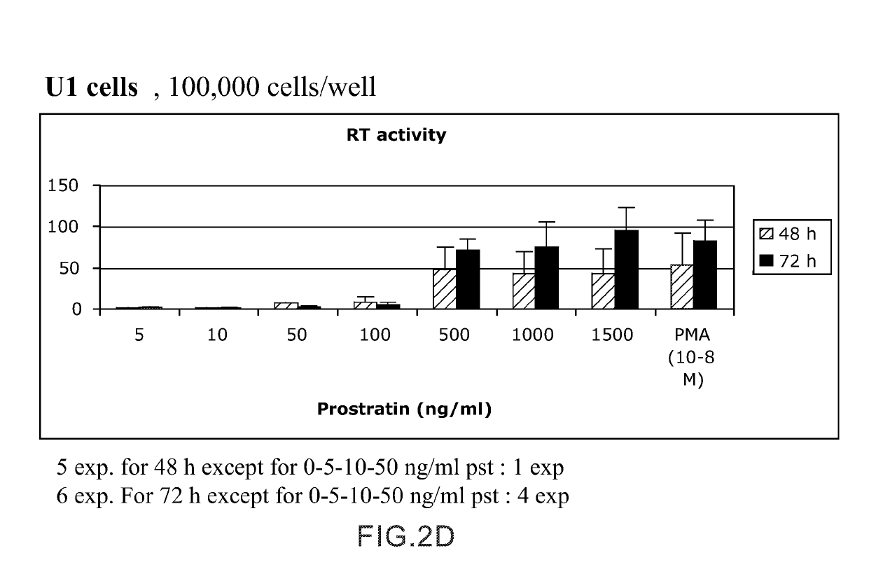

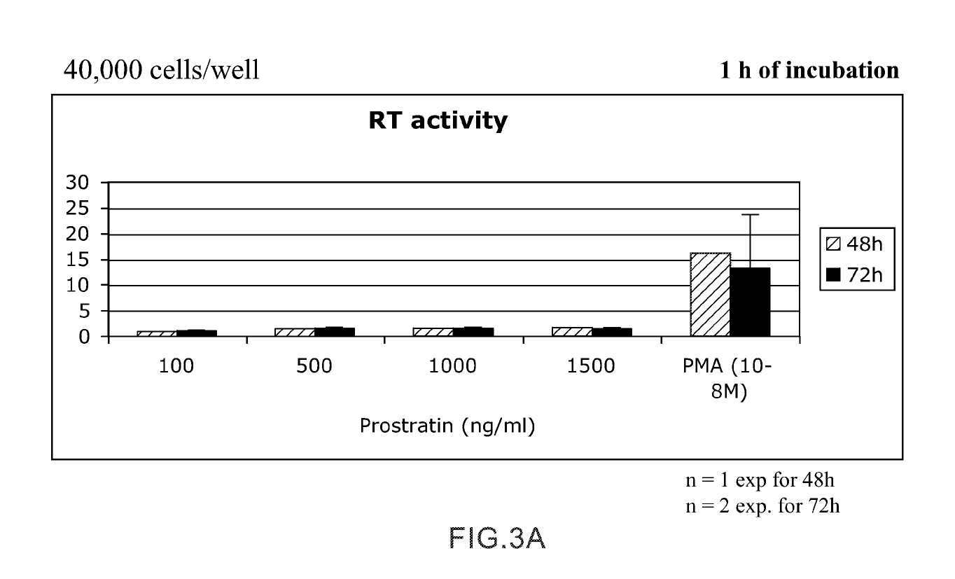

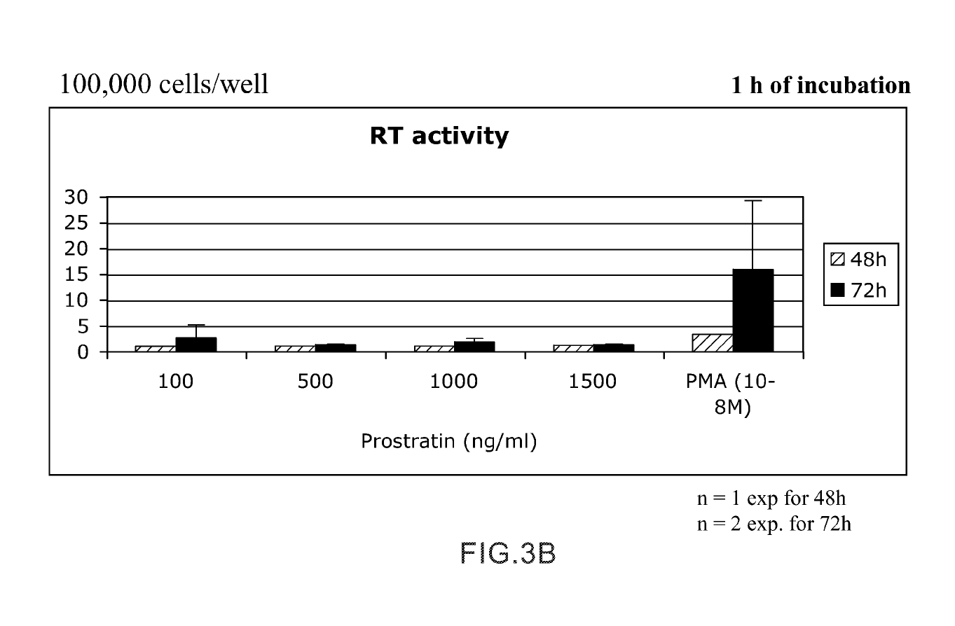

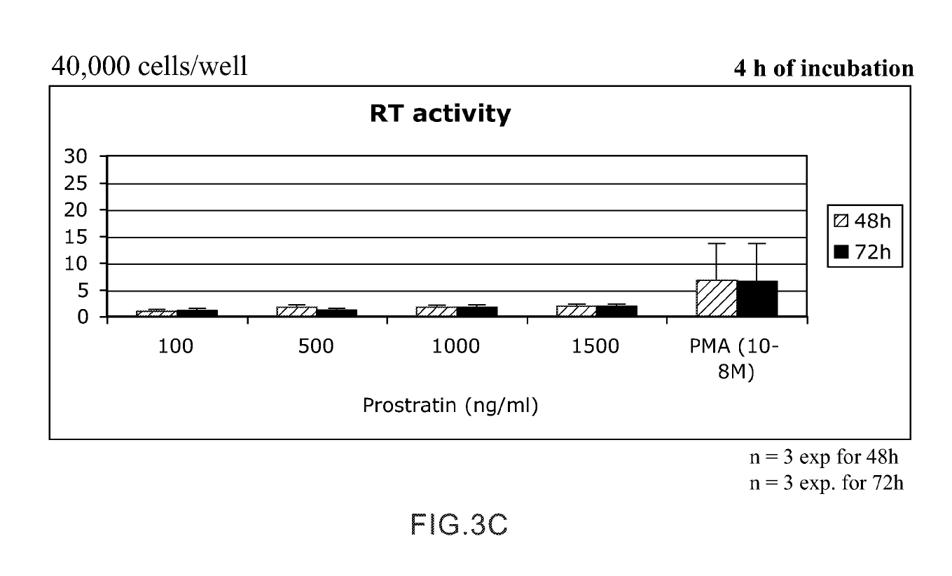

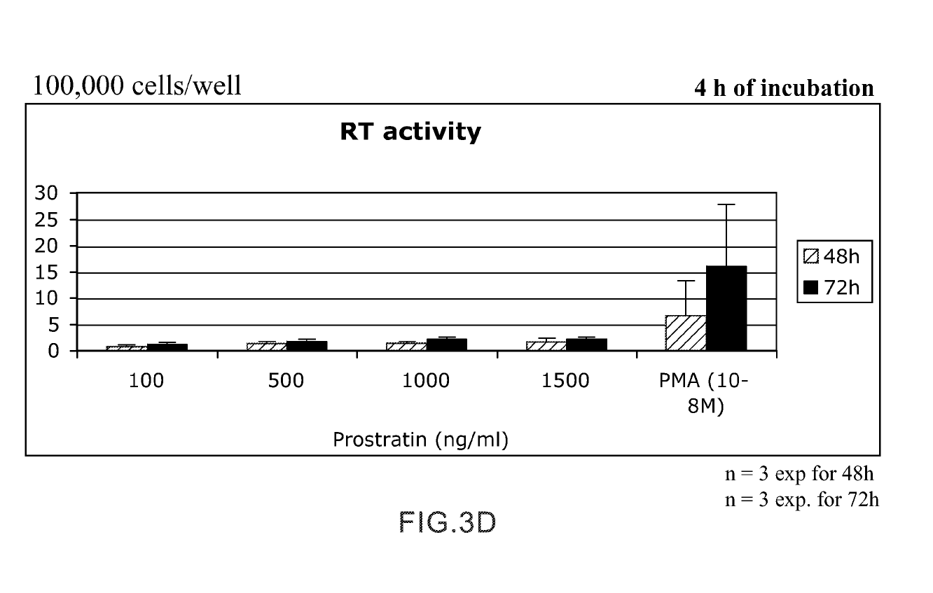

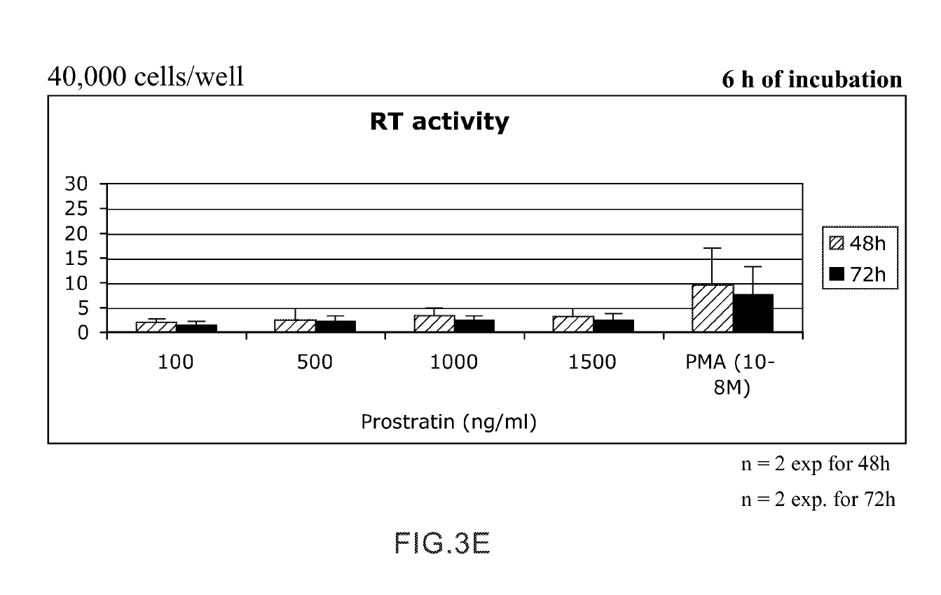

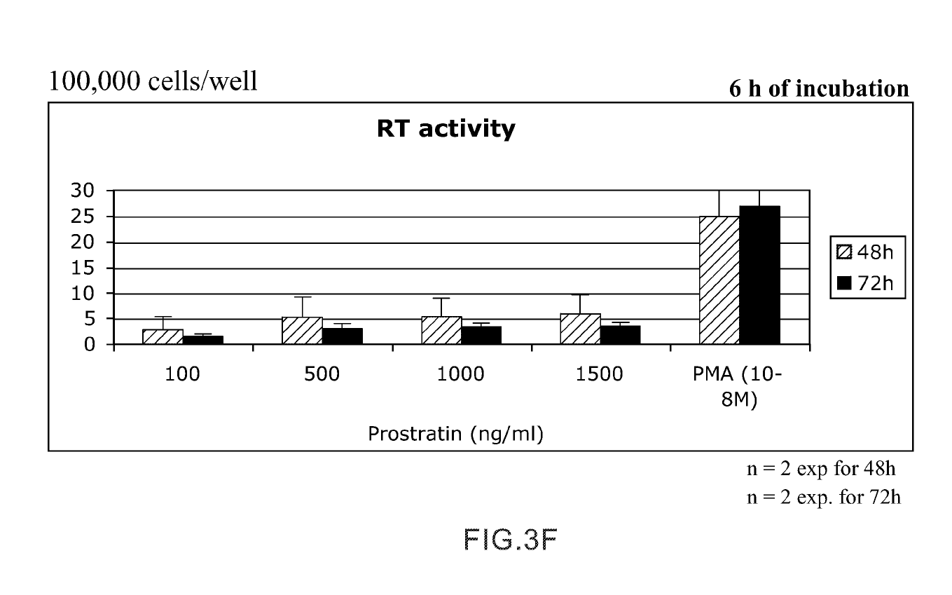

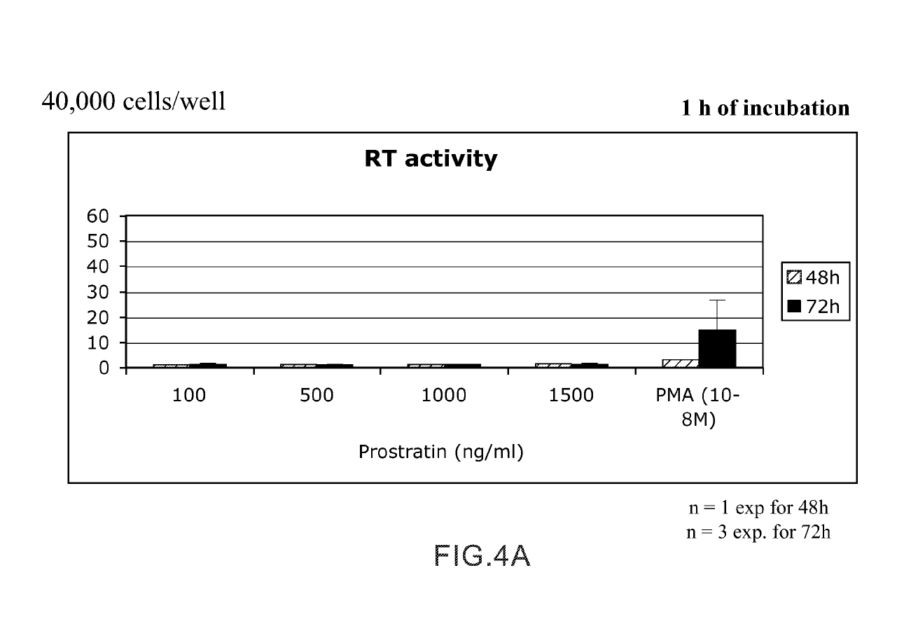

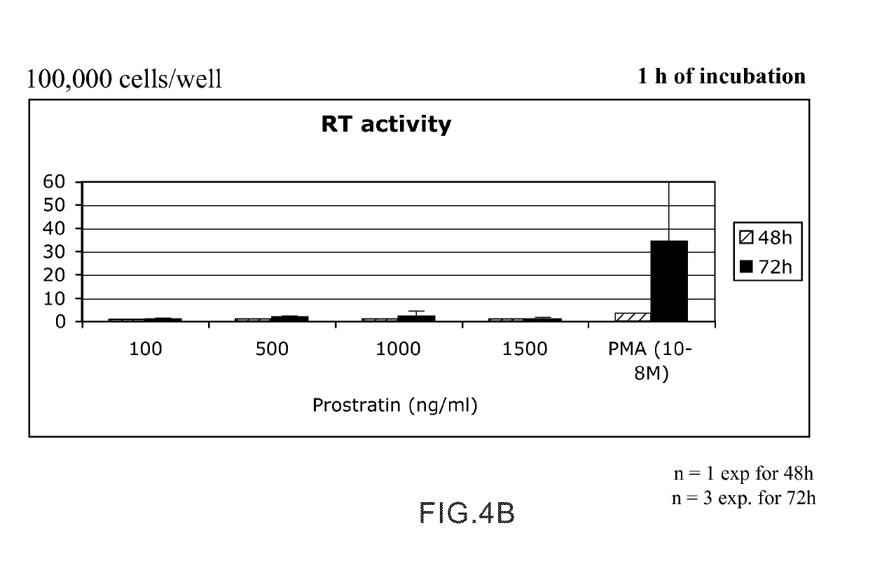

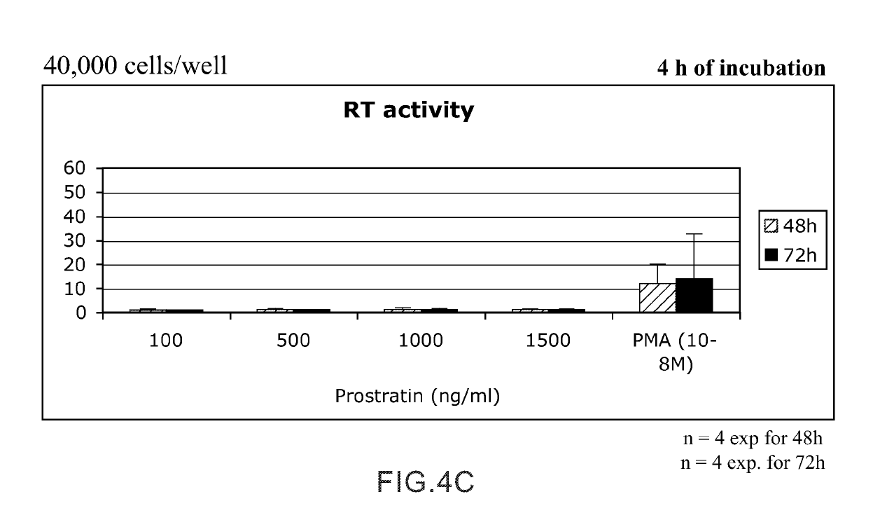

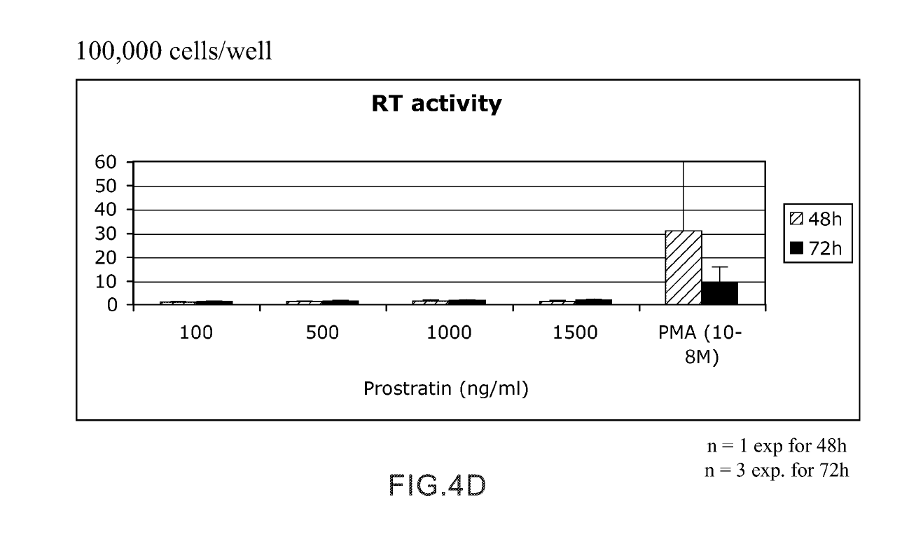

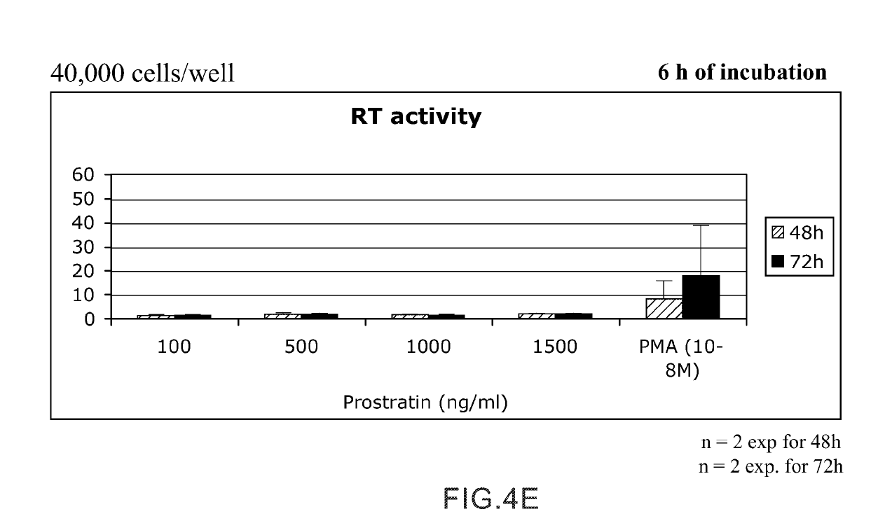

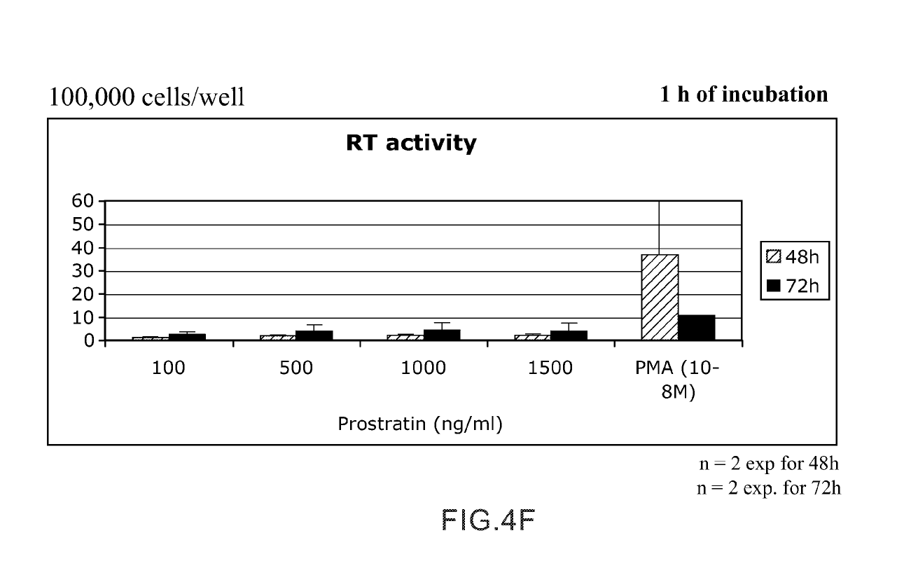

Analysis of data from these experiments suggested that the lowest dose of prostratin to get a reliable increase in reverse transcriptase activity in 50 ng/ml in ACH-2 cells and 50 ng/ml in U1 cells (FIG. 2). In addition, these experiments suggest that the apparent better sensitivity of U1 cells to prostratin in term of fold of increase following prostratin stimulation is due to the lower level of the basal reverse transcriptase activity in U1 cells as compared to ACH-2 cells. In term of absolute values, reverse transcriptase activity is always more elevated in ACH-2 cells as compared to U1 cells (FIG. 2). Moreover, the data suggest short incubations with prostratin led to a detectable reverse transcriptase activity only after 6 hours of prostratin treatment (no reverse transcriptase activity beyond an acceptable threshold level of detection is observed after 1 h and 4 h of pulsed stimulation in both cell lines) (FIGS. 2, 3). Lastly, a sizeable reverse transcriptase activity is already detected in ACH-2 cells after 24 h stimulation. U1 cells need to be exposed for at least 48 h to observe an acceptable reverse transcriptase activity (FIGS. 3, 4).

### Example 6

### Time- and Dose-Response Activity of Prostratin to Stimulate HIV Expression in PBMCs from HIV Positive Patients with Undetectable Plasma Viremia

The ability of prostratin to induce HIV-1 expression in cultures of highly purified resting CD4+ T cells derived from six HIV positive patients on HAART therapy with undetectable plasma viral load (<50 copies/ml) and CD4 cell counts above 350 cells/mm³ was evaluated. PBMCs were separated using Ficol Hypaque gradient and resting CD4+ T cells were purified using commercially available CD4+ T cell isolation kit (Miltenyi Biotec) Immediately after purification, resting CD4+ T cells were treated for 3 days with two antiretrovirals to which the patient had not previously been exposed. Antiretrovirals were used at concentrations 10 times the 50% inhibitory concentration for wild-type HIV-1 to ensure elimination of any actively replicating virus. Three days after in vitro incubation with antiretrovirals, cells were washed and incubated for 5 h, 24 h, or 48 h with prostratin (50-500 ng/ml), in the absence or presence of 1 mM valproic acid (sodium salt, SIGMA), 10 μg/ml PHA (SIGMA), 50 U IL-2/ml (AIDS depository of reagents, NIH), or in the absence of drugs. At the end of each incubation period, cells were co-cultured with CD8-depleted PBMCs from healthy donors. P24 was measured at days 0, 4, 7, 14, 21, 28, 35 and 42 days.

Our results confirm the ability of prostratin to induce HIV-1 expression in resting T cells. Ex-vivo treatment of resting T cells for 24 h with 150 ng/ml resulted in an efficient activation of the latent reservoir and led to high levels of HIV-1 expression. Robust HIV replication was observed after 2-4 weeks of co-culture with CD8-depleted PBMC, but not in mock-treated cultures. Lower concentration of prostratin (50 ng/ml) also resulted in HIV-1 activation events, although with lower frequency. Addition of VPA at 1 mM to cultures treated with prostratin did not significantly enhance HIV-1 replication beyond the prostratin effect. Importantly this study also showed patient-specific differences in the ability of resting T cells to become activated, ex-vivo.

### Example 7

### In Vitro Metabolism of ³H-Prostratin in Rat, Monkey, and Human Primary Hepatocytes

A previous study designed to understand the pharmacokinetic profile of prostratin indicated a difference in tolerability between male and female rats. The present study was conducted to examine whether there may be metabolic differences among rats, monkeys, and humans, or between genders. The metabolism of ³H-Prostratin was examined in both genders of rat, monkey, and human using isolated primary hepatocytes.

³H-Prostratin was incubated in hepatocyte preparations with 0.75×10⁶ hepatocytes/ml for 0, 30, 60, and 120 minutes. Hepatocytes were suspended in William's E medium and incubated with 1 μM or 4 μM ³H-Prostratin at 37° C. in an atmosphere of 4.7-4.8% CO₂. Hepatocyte incubations were terminated by addition of acetonitrile. Incubations were profiled for ³H-Prostratin and metabolites by high performance liquid chromatography using a radioactivity detector.

Metabolites were subsequently analyzed by liquid chromatography/tandem mass spectrometry (LC/MS/MS) for putative identification. Seven predominant metabolites, identified as M1 through M6 according to their chromatographic order of elution, were detected among both genders of the three species, including two co-eluting metabolites labeled as M3A or M3B. Among the seven metabolites, five were putatively identified by LC/MS/MS, while structures could not be proposed for M2 and M3B.

Seven metabolites were detected in hepatocyte incubations among both genders of rat, monkey, and human. Among the seven metabolites, five were identified by LC/MS/MS, while definitive structures could not be proposed for M2 and M3B. The five identified metabolites were deacyl-prostratin glucuronide (M1), deacyl-prostratin (M3A), prostratin glucuronide (M4), hydroxy prostratin No. 1 (M5), and hydroxy prostratin No. 2 (M6). For most metabolites, the specific site of metabolism could not be assigned due to the lack of sufficient fragmentation of prostratin by LC/MS/MS. However, hydroxylation was determined to have occurred on the acetyl group for M5. The extent of metabolism of ³H-prostratin was in the following rank order: male monkey>male rat>>female monkey≈male human≈female human>female rat. Male rat hepatocytes produced 2 unique metabolites, M2 and M5. No unique human metabolites were detected in the hepatocyte incubations.

A gender difference with respect to metabolism was observed in the rat, with the male rat exhibiting more rapid metabolism, formation of more and unique metabolites than female rats. Six metabolites of ³H-Prostratin (M2, M3A, M3B, M4, M5, and M6) were detected in male rat hepatocyte incubations and two (M3A and M6) by female rat hepatocytes.

Monkey hepatocytes of both genders produced five metabolites, but female monkey hepatocytes appear to have had a lesser ability to form M4 than male monkey hepatocytes. Because the male and female hepatocyte preparations were each obtained from a single individual, this difference could have resulted from individual genetic or environmental differences rather than a gender difference.

The metabolite profile of human hepatocyte incubations was identical to that of the female monkey. The extent of metabolism was considerably lower in human than in male monkeys.

The five putatively identified metabolites were deacyl-prostratin glucuronide (M1), deacyl-prostratin (M3A), prostratin glucuronide (M4), hydroxy prostratin No. 1 (M5), and hydroxy prostratin No. 2 (M6). For most metabolites, the specific site of metabolism could not be assigned due to the lack of sufficient fragmentation of prostratin by LC/MS/MS. However, hydroxylation was determined to have occurred on the acetyl group for M5.

There were no significant differences with respect to extent of metabolism or metabolic profile between the 1 μM and the 4 μM ³H-Prostratin incubations within each species and gender. The extent of metabolism of ³H-Prostratin by hepatocytes was in the following rank order: male monkey>male rat>>female monkey≈male human≈female human>female rat.

A metabolic gender difference was observed in the rat, with male rats exhibiting more rapid metabolism and formation of more metabolites than female rats. There were six total metabolites detected in male rat hepatocyte incubations (M2, M3A, M3B, M4, M5, and M6). Two metabolites unique to male rat hepatocytes were detected (M2 and M5). Female rat hepatocytes produced only two metabolites (M3A and M6).

Five metabolites (M1, M3A, M3B, M4, and M6) were produced by monkey hepatocytes and both genders of human hepatocytes. However, female monkey and human hepatocytes had considerably less capacity to form M4 than male monkey hepatocytes. Although the gender difference in rats for prostratin metabolism is likely to be real, caution should be taken in designating monkey as a species with a metabolic gender difference. Because male and female monkey hepatocyte preparations were each obtained from a single individual, the observed difference in M4 formation could have been due to individual genetic or environmental differences rather than a gender difference. The metabolite profile of human hepatocyte incubations was identical to that of monkey hepatocytes, although the extent of metabolism was considerably greater in the male monkey hepatocytes. There were no gender differences observed in human hepatocytes with respect to metabolism and there were no unique human metabolites.

### Example 8

### Salmonella-Escherichia coli/Mammalian-Microsome Reverse Mutation Assay with a Confirmatory Assay

The objective of this study was to evaluate the test article, prostratin, for the ability to induce reverse mutations either in the presence or absence of mammalian microsomal enzymes at 1) the histidine locus in the genome of several strains of Salmonella typhimurium and at 2) the tryptophan locus of Escherichia coli tester strain WP2uvrA.

The doses tested in the mutagenicity assay were selected based on the results of a dose range finding study using tester strains TA100 and WP2uvrA and ten doses of test article ranging from 6.67 to 5000 μg per plate, one plate per dose, both in the presence and absence of S9 mix.

The tester strains used in the mutagenicity assay were Salmonella typhimurium tester strains TA98, TA100, TA1535, and TA1537 and Escherichia coli tester strain WP2uvrA. The assay was conducted with five doses of test article in both the presence and absence of S9 mix along with concurrent vehicle and positive controls using three plates per dose. The doses tested were 100, 333, 1000, 3330, and 5000 μg per plate in both the presence and absence of S9 mix. The results of the initial mutagenicity assay were confirmed in an independent experiment.

The results of the Salmonella-Escherichia coli/mammalian-microsome reverse mutation assay with a confirmatory assay indicate that under the conditions of this study, the test article, prostratin, did not cause a positive increase in the mean number of revertants per plate with any of the tester strains either in the presence or absence of microsomal enzymes prepared from Aroclor™-induced rat liver (S9).

### Example 9

### Chromosomal Aberrations in Cultured Human Peripheral Blood Lymphocytes

The objective of this in vitro assay was to evaluate the ability of prostratin to cause structural chromosomal aberrations in cultured human lymphocytes with and without an exogenous metabolic activation system.

Dimethylsulfoxide (DMSO) was the vehicle of choice for this study. The highest dose used in the initial assay, 3500 μg/ml, was above the solubility limit of prostratin after dosing in culture medium. The stock solution and its dilutions were dosed using a dosing volume of 1% (10 μL/ml) and the vehicle control cultures were treated with 10.0 μL/ml of DMSO.

In the initial chromosomal aberrations assay, the treatment period was for 3 hours with and without metabolic activation, and cultures were harvested ˜22 hours from the initiation of treatment. Replicate cultures of human whole blood lymphocytes were incubated with test article at 23.7, 33.9, 48.4, 69.2, 98.9, 141, 202, 288, 412, 588, 840, 1200, 1720, 2450, and 3500 μg/ml with and without metabolic activation. Cultures treated with concentrations of 412, 588, 840, and 1200 μg/ml without metabolic activation and 588, 840, 1200, and 1720 μg/ml with metabolic activation were analyzed for chromosomal aberrations. No significant increase in cells with chromosomal aberrations, polyploidy, or endoreduplication was observed in the cultures analyzed.

In the confirmatory chromosomal aberrations assay, the treatment period was ˜22 hours without metabolic activation and 3 hours with metabolic activation, and cultures were harvested ˜22 hours from the initiation of treatment. Replicate cultures of human whole blood lymphocytes were incubated with test article at 15.6, 31.3, 62.5, 125, 188, 250, 375, 500, 750, 1000, 1400, and 1700 μg/ml without metabolic activation and 250, 500, 1000, 1400, 1700, 2000, and 2500 μg/ml with metabolic activation. Cultures treated with concentrations of 125, 250, 500, and 1400 μg/ml without metabolic activation and 1400, 1700, 2000, and 2500 μg/ml with metabolic activation were analyzed for chromosomal aberrations. No significant increase in cells with chromosomal aberrations, polyploidy, or endoreduplication was observed in the cultures analyzed.

For all treatment conditions, the vehicle and negative control cultures were within the expected range and the historical control data and the positive control cultures induced significant increases in chromosomal aberrations. The high doses selected for analysis had a precipitate at the end of the treatment period.

Prostratin was considered negative for inducing chromosomal aberrations in cultured human peripheral blood lymphocytes without and with an exogenous metabolic activation system.

### Example 10

### Pharmacokinetic Studies in Mice

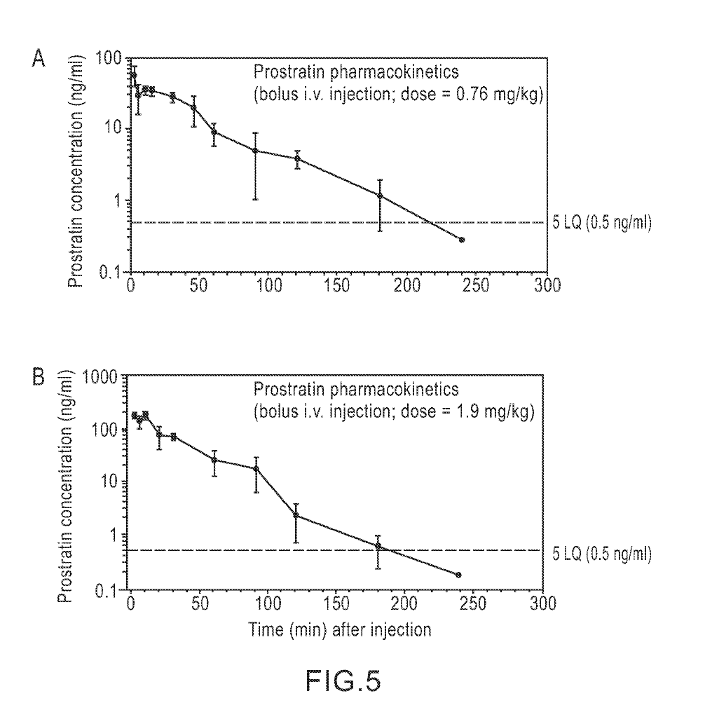

Studies were conducted in CD2F1 mice using both the i.p. route (1.9 mg/kg) and the i.v. route (0.76 mg/kg). The vehicle was 10% DMSO: 10% Cremophor: 80%:0.9% saline for both routes. Doses used were preciously determined as maximally tolerated. Drug was detected out to 4 hr but was only quantifiable out to 3 hr post drug administration (see FIG. 5A, B). Data are presented as mean±SD where n=5. Data that were detected beyond 3 hr post drug administration are shown as “BLQ” (below the limit of quantification of the validated assay). Data were fit using 1 and 2 compartment pharmacokinetic models using ADAPT II computer program. In general the 2-compartment model provided a better fit for the data than did a 1-compartment model (Table 2). Initial model fits using the single compartmental model demonstrated lower coefficients in variation for each PK parameter estimate but higher residual values for the observed vs. model predicted individual plasma concentration data. For the two-compartment model the situation was reversed (i.e. more variance for the model predicted PK parameters with lower residual values for the individual plasma concentration data). This suggests that a 2-compartment model would probably provide the best PK parameter estimates if sufficient plasma concentration-time data could be provided.

Prostratin is rapidly distributed as demonstrated by the i.p. plasma data (FIG. 5B) and cleared rapidly. On the order of 200-250 ml/min/kg for both the i.p and i.v. routes of administration. These data suggest that cells may rapidly internalize prostratin during the distribution period.

Urinary excretion of prostratin was measured in pooled samples from five mice dosed i.p. (1.9 mg/kg). Low levels of parent compound were detectable in urine collected at 6, 12, and 24 hours after dosing. Cumulative urinary excretion of prostratin was <5% of the administered dose.

TABLE 2
Pharmacokinetic Parameters for Prostratin in Mice.
Pharmacokinetic Parameter for Prostratin

| Route | Parameter | Estimate | CV (5) |
|---|---|---|---|
| IP (1.9 mg/kg) | CLt (L/hr/kg) | 13.15 | 2034 |
| | Vc (L/kg) | 11.03 | 3.04 |
| | T_{1/2} a (hr) | 0.34 | 4.22 |
| | T_{1/2} b (hr) | 210.2 | 4921 |
| | T_{1/2} (hr) | 0.34 | 2.85 |
| | [1-compartment model] | | |
| IV (0.76 mg/kg) | CLt (L/hr/kg) | 15.16 | 2314 |
| | Vc (L/kg) | 17.32 | 8.97 |
| | T_{1/2} a (hr) | 0.46 | 26.32 |
| | T_{1/2} b (hr) | 19.49 | 6101 |
| | T_{1/2 (hr)} | 0.53 | 8.02 |
| | [1-compartment model] | | |

### Example 11

### Single-Dose Range-Finding Study in Rats

A single bolus dose of prostratin (0, 0.05, 0.1, 0.2, 0.4, and 0.8 mg/kg which correspond to 0, 0.3, 0.6, 1.2, 2.4, and 4.8 mg/m²) was administered to Fisher rats (3 males/group) to determine the maximally tolerated dose and relative drug toxicity. All animals (3/3) in the 0.8 mg/kg dose group and two of the three animals in the 0.4 mg/kg dose group died on their Day 1 or Day 2. Clinical signs of toxicity included decreased activity, prostration, tremors, and altered respiratory rate (decreased in some cases, increased in others). On day 3, dose-dependent decreases in platelet (PLT) counts (62-68% decrease compared to group mean PLT counts for control rats) were seen in the 0.4 and 0.2 mg/kg dose groups, and a significant (1.7-fold) increase in neutrophile counts was noted in the 0.4 mg/kg dose group. In the 0.4 mg/kg dose group, ALT and AST serum levels were 2- and 3-fold higher, respectively, than group mean ALT and AST values for control rats, and potassium level was decreased in 61% of the value obtained for control group animals. Albumin (ALB) levels were dose-dependently decreased in the 0.4 and 0.2 mg/kg dose groups to 76% and 88%, respectively, of group mean ALB values for control group animals. By day 8, PLT, ALT, AST, and ALB levels were all within the normal range, indicating that the adverse events were reversible. The Maximum Tolerated Dose (MTD) in rats is between 0.2-0.4 mg/kg dose of prostratin.

### Example 12

### Dose-Range Finding Study in Monkeys and Determination of Maximum Tolerated Dose (MTD)

To determine plasma elimination kinetics and acute drug toxicity, a single intravenous bolus dose of either 0.1 or 0.4 mg/kg (1.2 or 4.8 mg/m²) of prostratin (formulated in ethanol/0.9% sodium chloride (0.8%/99.2%) were administered to rhesus macaque monkeys (1/sex/dose group). At day 3, 8, and 15 bloods were drawn for clinical pathology measurements. Additional blood was drawn in day 1 for plasma drug level determination, and on day 3 and 15 for analysis of cytokine (IL-2, IL-6, IL-8, GMCSF, and TNF-a). A maximum tolerated dose was not established in monkeys with the initial doses because of the absence of toxicity.

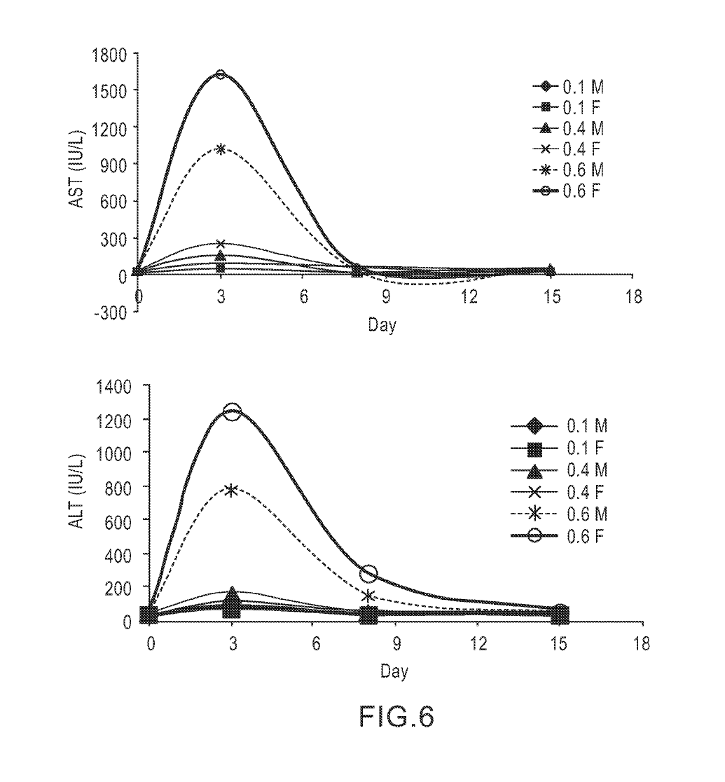

After a suitable wash-out period (15-20 days), monkeys that previously received 0.1 mg/kg, were administered a dose of 0.6 mg/kg, and those that previously received 0.4 mg/kg, were administered a second dose of 0.4 mg/kg. Blood was collected as before for clinical pathology and cytokine assay analysis. There was no mortality in this study. However both animals receiving 0.6 mg/kg prostratin required more time (2-4 times as much) to recover from Ketamine (used as a chemical restraint while weighing and dosing) after the second dosing compared with the initial doses. On day 3, all monkeys had elevated (2-3 fold) fibrinogen levels and there was evidence of dose-dependent hepatotoxicity. Animals in the 0.6 mg/kg dose group in particular exhibited severe hepatotoxicity with 25-50 fold increases in level of ALT and AST over baseline measurements (FIG. 6).

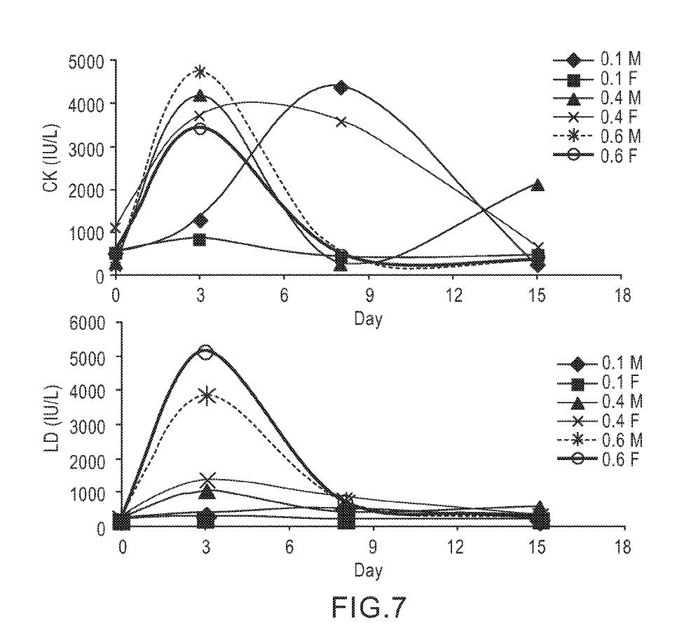

The marked clinical chemistry changes seen in the 0.6 mg/kg dose group does not appear to be the result of repeat dosing, since animals in the 0.4 mg/kg dose group also received 2 doses, yet higher levels of ALT, AST, CK, and LD were not seen after the second dosing. Additional changes in clinical pathology parameters include increases in CK and LD levels on day 3 (FIG. 7). These changes were dose-dependent even though the male monkey in the 0.1 mg/kg group achieved comparable CK level, because the onset of elevation was delayed 8 days compared to animals in the 0.4 mg/kg and 0.6 dose/groups that exhibited changes on day 3.

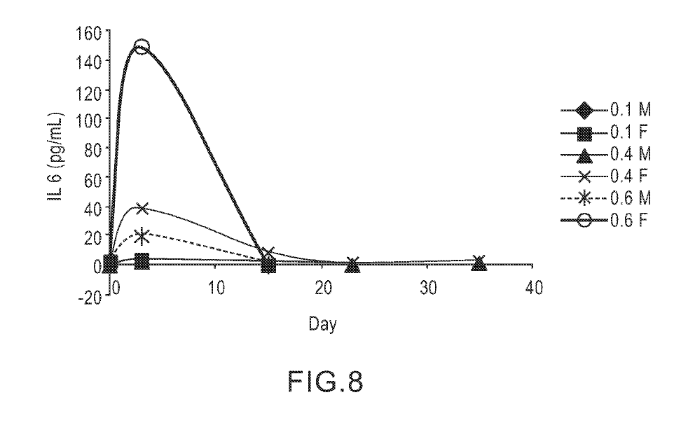

Analysis of various cytokines revealed an increase in the level of IL-6 in female monkeys that received 0.4 and 0.6 mg/kg prostratin (FIG. 8), suggesting an inflammatory response to drug treatment. All clinical pathology measurements were normal by day 15, indicating that these toxicities were reversible. The MTD in monkeys is between 0.4-0.6 mg/kg after a single bolus i.v. dose of prostratin.

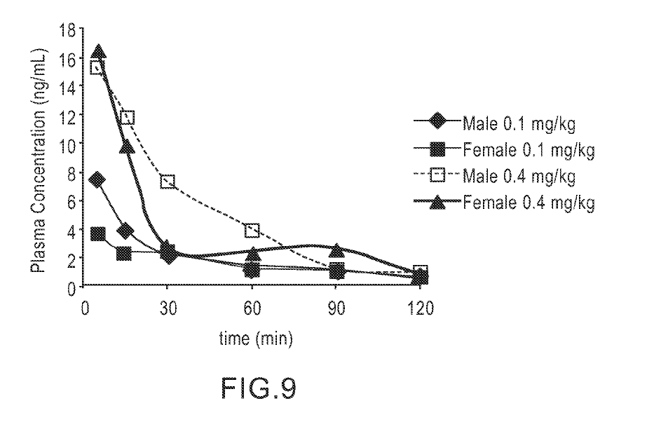

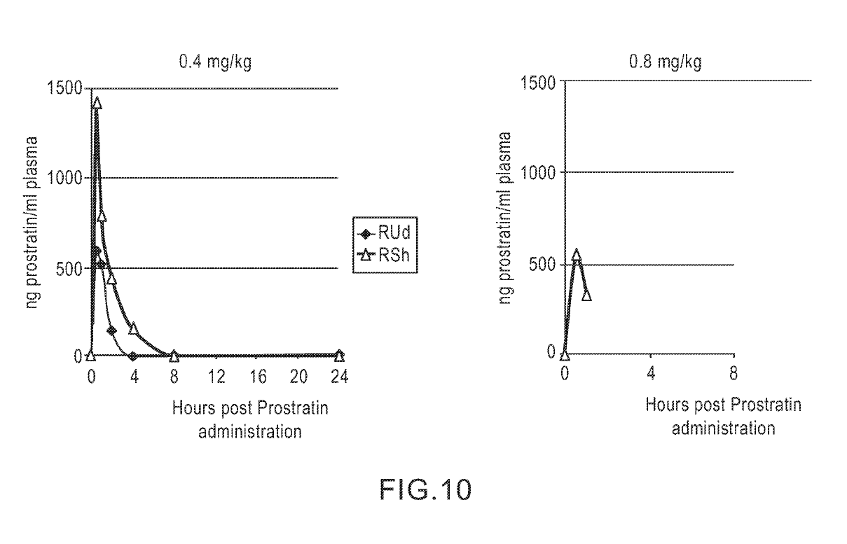

Determination of plasma drug level (0.1 and 0.4 mg/kg) at various time points after treatment on day 1 indicate that the drug is cleared rapidly from the blood and at these concentration is undetectable after 4 hours (FIG. 9).

### Example 13

### Pharmacology

### Mortality in Monkey

During a preliminary pharmacokinetic study, rhesus macaque monkeys were administered an intravenous bolus of prostratin at 0.4 mg/kg and 0.8 mg/kg. The monkey in 0.8 mg/kg died due a respiratory failure shortly after injection. Plasma samples from these monkeys were sent to NIH and plasma concentration of prostratin was measure along with other samples at NIH using the LC/MS/MS assay.

As shown in FIGS. 7 and 8, in the same dose group, plasma concentrations of prostratin in monkeys were much higher than the concentration in monkeys from NIH (used for dose range-finding studies, mentioned in section Example 12). The reason for this is unclear.

Furthermore, the dose of 0.8 mg/kg used was higher than the MTD later found monkeys (0.2-0.4 mg/kg) at NIH.

### Example 14

Determination of the Pharmacokinetics of Prostratin Following Intravenous and Oral Administration to Rats

In order to find out the best route of administration and the correct concentration of prostratin, the drug was administered as an intravenous infusion over three hours as well as a single and twice oral administration to 3 groups of rats. The goal of this experiment was to find out if greater amounts of prostratin can be administered over a longer period of time. This would enable obtaining median higher concentrations in the blood for a longer period of time that would be close to effective concentration found in in vitro studies.

Study Design

TABLE 3
Study Design for Dosages of Prostratin

| | | | | | Target | Target | Target |
|---|---|---|---|---|---|---|---|
| | Number of | | | | Dose | Dose | Dose |
| | | Fe- | Test | Dose | Level | Conc. | Volume |
| Group | Males | males | Article | Route | (mg/kg) | (mg/ml) | (ml/kg) |
| 1 | 3 | 3 | Prostratin | I.V. | 0.8 | 0.16 | 5 |
| 2 | 3 | 3 | Prostratin | Oral | 2.4 | 0.48 | 5 |
| 3 | 3 | 3 | Prostratin | Oral | 1.8 | 0.36 | 5 |

Note:
Animals in Group 3 received 2 oral doses (values listed are for each dose); the second dose was given approximately 4 hours after administration of the first dose.
I.V., Intravenous; given as an approximate 3-hour infusion via a femoral vein cannula.

Dosage

Extrapolation was performed from the DART work that was all done in rats as an IV injection (not infusion). Infusions and injections are by definition 100% bio-available meaning it all gets in.

The oral doses were estimated based on a maximum tolerated i.v. dose of 0.4 mg/kg from the DART work in rats, but an oral bio-availability of only 29%. Thus, the maximum tolerated i.v. dose was multiplied by three (i.e., 0.4 mg/kg times 3 equals 1.2 mg/kg as the equivalent oral dose). The purpose of the two lower oral doses (both 0.8 mg/kg, therefore a total of 1.6 mg/kg) and the infusion (0.8 mg/kg over three hours) was to see if the maximum tolerated dose could be exceeded by dividing up the oral into two times 75% the max tolerated dose or as an infusion over 3 hours.

Prostratin was tolerated by the male animals (Table 4). However, the dams all died after intravenous and both single and double oral administration of prostratin (Table 5).

Gender differences in the study were explored and identified as related to different gender influenced metabolism (see Example 7). One male rat did die in the infusion but it was attributed to a problem with the jugular catheter.

TABLE 4
Dosages in Male Rats (MTD via iv bolus dose: 0.4 mg/kg)

| | Male rats | Doses | Route of | |
|---|---|---|---|---|
| Groups | (number) | (mg/kg) | administration | Results |
| 1 | 3 | 0.8 | I.V. infusion, 3 | All animals tolerated but |
| | | | hours | one death attributed to |
| | | | | catheter problem |
| 2 | 3 | 2.4 | One oral dose | All animals are alive |
| 3 | 3 | 1.8 | Two oral doses | All animals are alive |
| | | | 4 hours apart | |

TABLE 5
Dosages in Female Rats

| | Female | | | |
|---|---|---|---|---|
| | rats | Doses | Route of | |
| Groups | (number) | (mg/kg) | administration | Results |
| 1 | 3 | 0.8 | I.V. infusion, 3 | All animals died |
| | | | hours | |
| 2 | 3 | 1.2 | One oral dose | 2 out of 3 animals died |
| | | | | immediately, one at 3 hrs |
| 3 | 3 | 1.8 and | 1.8 mg/kg first | 2 died after first dose, |
| | | 1.0 | oral dose, | one later |
| | | | followed by a | |
| | | | second dose of | |
| | | | 1.0 mg/kg after | |
| | | | 4 hours | |

Note: The dosage in Groups 2 and 3 were decreased in female rats compared to Table 1, because of the fact that all animals died in Group 1.

Following a single 0.8-mg/kg intravenous infusion dose of prostratin over 3 hours, a mean Cₘₐₓ of 159 ng/ml was observed; Cₘₐₓ was observed at the end of the infusion for two male rats and at 5 minutes following completion of the infusion for the remaining male rat. Calculated plasma concentrations and pharmacokinetics varied markedly between male and female rats in this dose group. Two of the three female rats were euthanized following the blood collection at approximately 2 hours from the start of the infusion due to moribund conditions. The concentration of prostratin in plasma for the two females at this time point was approximately 3.8-fold (Animal No. C24047) and approximately 1.6-fold higher (Animal No. C24048) than the mean Cₘₐₓ observed for the males. Analysis of exposure to prostratin for 2 hours from the start of the infusion (AUC₀₋₂) showed that the two females were approximately 4.2-fold (Animal No. C24047) and approximately 1.4-fold higher (Animal No. C24048) than the mean AUC₀₋₂ for the males. The remaining female (Animal No. C24049) survived the 3-hour infusion dose, but was euthanized following the blood sample collection at 3.083 hours postdose due to poor health. The Cₘₐₓ and AUC_{0-3.083} for the remaining females were approximately 3.7- and 2.6-fold higher, respectively, than the corresponding mean values for the males.

Following completion of the 3-hour intravenous infusion for the males, concentrations of prostratin steadily declined with a mean t_{1/2} of 2.49 hours. The mean V_{z}, Vₛₛ, and CL for prostatin in male rats were 6409 ml/kg, 2218 ml/kg, and 2055 ml/hr/kg, respectively. Due to insufficient correlation of data characterizing the elimination phase for one male (Animal No. C24038), calculation of the elimination phase half-life (t_{1/2}) and parameters obtained via extrapolation to infinity was unable to be performed. Notable variation was observed between the pharmacokinetic parameters (t_{1/2}, V_{z}, Vₛₛ, and CL) for the two males; as a result, the mean pharmacokinetic data should be viewed with some caution. Animal No. C24038 died on test during the blood sample collection at approximately 7 hours postdose (a sample was unable to be obtained); the exposure to prostratin through 5 hours postdose (AUC₀₋₅) for this animal was similar to the other two males, which supported the hypothesis that the animal's mortality was probably due to unusual complications with the jugular vein cannula instead of circulating levels of prostratin.

Following a single 2.4-mg/kg oral dose of Prostratin to male rats, a mean Cₘₐₓ of 49.7 ng/ml was observed in the range of 0.25 to 0.5 hours postdose. Female rats in this dose group each received a single 1.2-mg/kg oral dose due to observations seen in females following the first 1.8-mg/kg oral dose in Group 3 (see subsequent section below, BID Oral Administration). One female rat in Group 2 (Animal No. C24050) was euthanized at 1.5 hours postdose following the scheduled blood sample collection. The Cₘₐₓ and AUC_{0-1.5} for this female were approximately 8.9- and 8.2-fold higher, respectively, than the corresponding mean values for the males. The mean Cₘₐₓ and mean AUC_{0-1.5} for the all three females were approximately 6.2- and 5.5-fold higher, respectively, than the corresponding mean values for the three males. A second female rat (Animal No. C24052) was euthanized following the blood sample collection at 4 hours postdose. The AUC₀₋₄ for Animal No. C24052 was approximately 9.6-fold higher than the corresponding mean value for the males. The last female rat survived to study completion at 10 hours postdose, and the AUC₀₋₁₀ for this female was approximately 6.1-fold higher than the mean AUC₀₋₁₀ value for the males. AUC_{0-∞} and t_{1/2} were unable to be calculated for the last female due to insufficient correlation of data characterizing the elimination phase.

After reaching Cₘₐₓ in male rats following the single oral dose, concentrations of Prostratin steadily declined with a mean t_{1/2} of 2.08 hours. Pharmacokinetic parameters were similar for two of the three males in this dose group, with the third animal displaying higher oral absorption of Prostratin. Bioavailability (% F) of Prostratin following the single 2.4-mg/kg oral dose to the three male rats ranged from 7.7% to 23.1% (mean 13.3%±8.5%).

Twice-daily oral administration of Prostratin (second oral dose given 4 hours after the first oral dose) was investigated over the course of 1 day in this study in an effort to determine preliminary accumulation effects and any changes in pharmacokinetic parameters in comparison to the single oral dose group which received a slightly higher dose (Group 2). Following single 1.8-mg/kg oral doses of Prostratin to male rats in Group 3, a mean Cₘₐₓ of 55.1 ng/ml was observed in the range of 0.25 to 0.5 hours following the second dose. The mean plasma concentration of Prostratin for the males just prior to the second oral dose at 4 hours was 17.9 ng/ml, which was similar to the mean plasma concentration (11.5 ng/ml) of Prostratin in males at 4 hours postdose following a single 2.4-mg/kg oral dose in Group 2. After reaching Cₘₐₓ in male rats following the second oral dose, concentrations of Prostratin steadily declined with a mean t_{1/2} of 4.07 hours, although two of the three males had t_{1/2} values that were similar to the mean t_{1/2} value obtained for Group 2. The third animal in Group 3 (Animal No. C24046) had a t_{1/2} value of 7.47 hours. In similar fashion to Group 2, some inter-animal variability was observed between the pharmacokinetics parameters for the three males comprising Group 3.

Based on comparison of mean AUC₀₋₁₀ values for male rats in Groups 2 and 3, the 1.8-mg/kg BID dosing regimen yielded an approximately 1.5-fold increase in exposure to Prostratin following the second daily dose over the course of 1 day in comparison to male rats that received a single 2.4-mg/kg oral dose.

The three female rats in Group 3 each received a single 1.8-mg/kg oral dose initially. One female (Animal No. C24053) was euthanized at approximately 1.5 hours postdose, and another female (Animal No. C24054) was euthanized at approximately 2 hours postdose. Blood samples were collected from each of these two females just prior to sacrifice. Concentrations of Prostratin in plasma for these samples were similar to the concentrations observed at these sampling time points following the single 1.2-mg/kg oral dose of Prostratin to the female rats in Group 2. The remaining female rat (Animal No. C24055) survived the initial 1.8-mg/kg oral dose with notable adverse observations seen, but the second oral dose given 4 hours after the first dose was lowered to 1.0 mg/kg given the findings from the first dose. Animal No. C24055 survived through the sample collection at 4 hours postdose (based on the time at which the second oral dose was given), and then the animal was euthanized due to the adverse observations seen. The Cₘₐₓ, AUC_{0-1.5}, and AUC₀₋₄ values for the female were approximately 6.0-, 5.9-, and 8.2-fold higher, respectively, than the corresponding mean values for the three males.

Marked gender-related differences with respect to tolerability to prostratin were observed across all 3 dose regimens that were investigated. The concentration of prostratin in plasma of female rats after both intravenous and oral administration of prostratin was several folds higher than in male rats, notwithstanding identical administered doses.

Male rats were able to tolerate prostratin at the doses investigated in this study markedly better than female rats. Only one of the female rats (Group 2) was able to complete the study, other females were euthanised due to the severity of the moribund observations.

All but one of the male rats survived to study completion. The male rat (Animal No. C24038) in the 3-hour intravenous infusion dose group (Group 1) died on test during an attempt to obtain a blood sample at approximately 7 hours following infusion initiation. The in-life technician had observed that there was some unusual difficulty in utilizing the jugular vein cannula for blood sample collections for this particular animal. Given the observations obtained for this animal in comparison to the other two males in this dose group and the fact that the jugular vein cannula use was unusually difficult, the animal's fatality was not attributed to circulating levels of prostratin.

### Example 15

### Quantitative Whole Body Autoradiography of Rats Following Oral Administration of ³H-Prostratin

Because a previous study designed to evaluate the pharmacokinetic profile of the drug confirmed the difference in tolerability between male and female rats, this absorption and distribution study was conducted to further evaluate the observed gender difference.

The absorption and distribution of ³H-Prostratin were studied in male and female rats following a single oral administration at 0.2 mg/kg. Four Long Evans male and four Long Evans female rats, after an overnight fast, each received a single oral administration of ³H-Prostratin as a solution in saline containing <1% of ethanol at a target dose of 0.2 mg/kg. For both males and females, one animal per time point per sex was sacrificed at 1, 4, 8, and 24 hours postdose and carcasses were prepared for whole-body autoradiography (WBA). Blood, plasma, and cellular fraction were analyzed by using liquid scintillation counting (LSC).

The group designation, number of animals, target dose level, and target dose volume were as follows:

TABLE 6
Group Designations and Dose Levels

| | | | | Target | Target | |
|---|---|---|---|---|---|---|
| | Number | | | Dose | Dose | |
| | of Animals | | Dose | Level | Volume | |
| Group | Male | Female | Route | (mg/kg) | (ml/kg) | Samples Collected |
| 1 | 4 | 4 | Oral | 0.2 | 5 | Carcasses for |
| | | | | | | WBA and Blood |

WBA Whole-body autoradiography.
Note:
The radioactive dose was approximately 300 μCi/kg.

Following an oral administration of ³H-Prostratin to rats, the maximum blood and plasma concentrations of radioactivity in males were 30.5 and 53.5 ng equivalents ³H-Prostratin/g, observed at 1 and 4 hours postdose, respectively. In females, the maximum blood and plasma concentrations of radioactivity were 59.7 and 63.7 ng equivalents ³H-Prostratin/g, respectively, observed at 1 hour postdose. The concentration of radioactivity in blood and plasma for both males and females slowly declined through 24 hours postdose.

After oral administration to male and female rats, ³H-Prostratin-derived radioactivity was distributed to a limited number of tissues. Most of the radioactivity was found in the gastrointestinal (GI) tract in both males and females. In males at 1 hour postdose, radioactivity was detected in bile, liver, urine, and GI tract. In females at 1 hour postdose, excluding GI tract, radioactivity was found in adrenal gland, bile, liver, myocardium, ovary, preputial gland, thymus, and urine. At 24 hours postdose, radioactivity was only detected in large intestine contents and cecum contents in both males and females.

Based on these data, it appears ³H-Prostratin-derived radioactivity was distributed more widely in female tissues than male tissues, suggesting a gender-related difference. These data support the earlier finding of a difference between male and female rats.

Radioactivity in male and female plasma steadily declined over the course of the study. The percentages of tritiated water in plasma increased over time both in males and females.

³H-Prostratin-derived radioactivity was distributed to a limited number of tissues in both males and females, mostly in the GI tract. Radioactivity was distributed more widely in female tissues than male tissues, suggesting a gender-related difference.

### Example 16

### In Vivo Rat Bone Marrow Micronucleus Assay

³H-Prostratin-derived radioactivity was distributed to a limited number of tissues in both males and females, mostly in the GI tract. Radioactivity was distributed more widely in female tissues than male tissues, suggesting a gender-related difference.

The objective of this study was to evaluate the test article, prostratin, for in vivo clastogenic activity and/or disruption of the mitotic apparatus by detecting micronuclei in polychromatic erythrocytes (PCE) in CD® (SD) IGS BR rat bone marrow.

In the dose range-finding study, the test article was dissolved in ethanol followed by dilution with 0.9% Sodium Chloride for Injection to achieve a final ethanol:saline vehicle (0.8:99.2 v/v) at the appropriate dosing concentrations. The formulations were administered once by oral gavage to three males and three females per dose level. The animals were dosed at 0.4, 0.5, 2, or 5 mg/kg and observed for up to 2 days after dosing for toxic signs and/or mortality.

Based on the results of the dose range-finding study, the maximum tolerated dose was estimated to be 2.4 mg/kg in the male animals and 2 mg/kg in the female animals. In the micronucleus assay, the test article was dissolved in ethanol followed by dilution with 0.9% Sodium Chloride for Injection to achieve a final ethanol:saline vehicle (0.8:99.2 v/v) at the appropriate dosing concentrations.

The formulations were administered once, as follows:

TABLE 7
Administration Scheme

| Target | Stock | Dosing | | Animals/Harvest Timepoint | | | | Replacement | |
|---|---|---|---|---|---|---|---|---|---|
| Dose Level | Concentration | Volume | Route of | 24 Hour | | 48 Hour | | Animals^{a} | |
| (mg/kg) | (mg/ml) | (ml/kg) | Administration | Male | Female | Male | Female | Male | Female |
| Positive Control, 60 | 6 | 10 | Oral Gavage | 5 | 5 | — | — | — | — |
| Vehicle Control, 0 | 0 | 10 | Oral Gavage | 5 | 5 | 5 | 5 | — | — |
| 0.6 | 0.06 | 10 | Oral Gavage | 5 | — | — | — | — | — |
| 1.2 | 0.12 | 10 | Oral Gavage | 5 | — | — | — | — | — |
| 2.4 | 0.24 | 10 | Oral Gavage | 5 | — | 5 | — | 5 | — |
| 0.5 | 0.05 | 10 | Oral Gavage | — | 5 | — | — | — | — |
| 1 | 0.10 | 10 | Oral Gavage | — | 5 | — | — | — | — |
| 2 | 0.20 | 10 | Oral Gavage | — | 5 | — | 5 | — | 5 |

Vehicle Control = Ethanol:saline vehicle (0.8:99.2 v/v),
Positive Control = Cyclophosphamide
^{a}Animals were dosed as potential replacements for the original high-dose groups.

Bone marrow was extracted and at least 2000 PCEs per animal were analyzed for the frequency of micronuclei. Cytotoxicity was assessed by scoring the number of PCEs and normochromatic erythrocytes (NCEs) in at least the first 500 total erythrocytes for each animal.

The test article, prostratin, induced signs of clinical toxicity in the treated animals at 2 mg/kg in the female animals approximately one hour post-dose, which included mortality, irregular respiration, piloerection, and/or recumbency. All remaining animals were normal by the next morning. Prostratin did not induce statistically significant increases in micronucleated PCEs at any test article dose examined (0.5, 1, or 2 mg/kg for females or 0.6, 1.2, and 2.4 mg/kg for males). In addition, prostratin was not cytotoxic to the bone marrow (i.e., no statistically significant decreases in the PCE:NCE ratios) at any dose of the test article.

The test article, prostratin, was evaluated as negative in the rat bone marrow micronucleus assay under the conditions of this assay.

### Example 17

### In Vitro Tests Measuring the Ability of Prostratin Analogs to Activate HIV Replication in Viral Reservoirs

### 1. Screening of Prostratin Analogs in Latently Infected Clonal Cells Including Lymphocytic and Monocytic ACH₂ and U1 Cells

The effect of prostratin analogues on virus replication is assessed in latently infected ACH-2 and U1 cells by measuring the accumulation of p24 in the supernatant of stimulated cells. ACH-2 cells (latently infected with HIV-1_{LAV}) and U1 cells (latently infected with HIV-1) are cultured at 37° C./5% CO₂ in appropriate medium. Following incubation of cells with various concentration of prostratin analogues, supernatants are harvested, and HIV-1 production is determined with a p24 antigen ELISA kit following the manufacturer's instructions (Zeptometrix).

2. Screening of Prostratin Analogs in Resting CD4+ Cells from Patients Under Treatment with Undetectable Plasma Viremia (Viral Load <50 copies/ml)

Blood (50-100 ml) from HIV-1-seropositive individuals (anti-retroviral treated, viral load <50 copies/ml) is used for isolation of Peripheral Blood Mononuclear Cells (PBMC). Resting CD4+ cells are isolated from PBMCs using a commercially available CD4+ T cell isolation kit (Miltenyi Biotec) following the manufacturer's recommendations. This kit depletes PBMCs from cells expressing the following surface expression proteins: CD8, CD14, CD16, CD19, CD36, CD56, CD123, TCRg/d and glycophorin A. Recovered resting CD4+ cells are stained with fluorochrome-conjugated antibodies and analyzed by flow cytometry in a LSRII Becton-Dickinson apparatus. Immediately after purification, resting CD4+ T cells are treated for 3 days with two antiretrovirals (ARVs) to which the patient had not previously been exposed. ARVs are used at 10 times the 50% inhibitory concentration for wild-type HIV-1 to ensure elimination of any actively replicating virus. Following treatment with ARV, cells are washed twice and incubated for 5 h, 24 h, or 48 h with prostratin analogues (50-500 ng/ml), 10 μg/ml PHA (SIGMA), 50 u IL-2/ml (AIDS depository of reagents, NIH), or in the absence of protratin analogues. After treatment, cells are co-cultured with CD8-depleted PBMC from healthy donors.

The effect of prostratin analogues on virus replication in this system is assessed by measuring the accumulation of P24 (p24 antigen ELISA kit) in the supernatant of stimulated cells at various time points.

### 3. In Vitro Latency Model Using Green Fluorescent Protein-Luciferase Fusion Protein-Containing Reporter Virus

In this assay, transcriptionally active immature CD4+ CD8+ thymocytes are infected with HIV, and a latent infection develops as these cells convert to the less transcriptionally active mature CD4+ or CD8+ thymocytes. Transcriptionally active immature CD4+ CD8+ thymocytes are infected with NLEGFPLuc, a vector that contains a gene encoding an EGFP-luciferase fusion protein in place of nef that is expressed under the control of the HIV-LTR. Cells are then incubated with various concentrations of prostratin analogues at various time points. The effect of prostratin analogues on virus replication is assessed by measuring luciferase activity using a luminometer. The effect of prostratin analogues on virus replication in this system is assessed by measuring the accumulation of P24 (p24 antigen ELISA kit) in the supernatant of stimulated cells at various time points.

4. In vitro Latency Model Using Peripheral Blood Mononuclear Cells Transfected with Luciferase Fusion Protein-Containing Reporter Virus

In this assay, peripheral blood mononuclear cells (PBMCs) are transfected with luciferase expression constructs under the control of wild type HIV-LTR and consensus sequences for transcription factors involved in HIV-LTR transactivation (NF-κB, SP1, NFAT). Cells are incubated with prostratin analogues at various concentrations and their ability to stimulate transactivation of LTR vectors, κB- and SP-1-driven luciferase constructs is assessed by measuring luciferase activity.

In another sets of assays, PBMCs are transfected with a full-length infectious HIV clone. The ability of a prostratin analog to stimulate HIV transcription and viral expression is detected by luciferase activity in cellular extracts and p24 levels in culture supernatents, respectively.

5. In vitro Latency Model Using Ex Vivo Infected Human Tonsil Tissues

In this assay, human tonsil tissues are infected with HIV-1 NL4-3. The effects of various concentrations of prostratin analogs in stimulating viral replication are assessed by analyzing p24 accumulation in the culture medium.

The present invention has been described with reference to specific details of particular embodiments thereof. It is not intended that such details be regarded as limitations upon the scope of the invention except insofar as and to the extent that they are included in the accompanying claims. All patents and publications cited are herein incorporated in their entireties for all purposes.

What is claimed is:

1. A method for inducing latent HIV-1 expression in a mammalian cell, the method comprising administering to a mammal in need thereof a dosage amount of about 2.5 μg/kg/hr to about 50 μg/kg/hr of prostratin or a structural analog or metabolite thereof.

2. The method of claim 1, wherein prostratin or a structural analog or metabolite thereof is administered by infusion.

3. The method of claim 2, wherein prostratin or a structural analog or metabolite thereof is administered for about 2 hours to about 72 hours.

4. The method of claim 3, wherein prostratin or a structural analog or metabolite thereof is administered for about 4 hours to about 24 hours.

5. The method of claim 4, wherein prostratin or a structural analog or metabolite thereof is administered by infusion for about 6 hours.

6. The method of claim 5, which comprises administering to said patient about 5 μg/kg/hr to about 15 μg/kg/hr of prostratin or a structural analog or metabolite thereof.

7. The method of claim 1, wherein prostratin or a structural analog or metabolite thereof is further administered with a pharmaceutically acceptable carrier, excipient, or diluent.

8. The method of claim 2, wherein administration by infusion is selected from the group consisting of intravenous, intraarterial, intralymphatic, and intraperitoneal administration.

9. The method of claim 2, wherein prostratin or a structural analog or metabolite thereof is administered using a pump.

10. The method of claim 1, wherein said mammal is a human.

11. The method of claim 10, further comprising the step of administering HAART.

12. The method of claim 1, wherein the mammalian cell is a resting lymphoid mononuclear cell.

13. The method of claim 12, wherein the resting lymphoid mononuclear cell is a CD4+ T cell.

14. A kit for inducing latent HIV-1 expression in a mammalian cell, comprising prostratin or a structural analog or metabolite thereof packaged with instructions for infusing prostratin or a structural analog or metabolite thereof to induce latent HIV-1 expression.

15. The kit of claim 14, further comprising a pharmaceutically acceptable carrier, excipient, or diluent.

16. The kit of claim 14, wherein prostratin or a structural analog or metabolite thereof is in a form suitable for infusion.

17. The kit of claim 14, wherein prostratin or a structural analog or metabolite thereof is in a form suitable for intravenous, intraarterial, intralymphatic or intraperitoneal administration.

18. A method for inducing latent HIV-1 infection in a mammalian cell, the method comprising administering prostratin, a prostratin prodrug, or a structural analog thereof or metabolite thereof as an orally active sustained release formulation to a mammal in need thereof.

19. The method of claim 18, wherein the sustained release formulation is administered orally in tablet form at least once, twice, or three times over a 24 hour period.

20. The method of claim 19, wherein the effective plasma concentration attained by the sustained release formulation is sustained for at least 4 hours.

21. The method of claim 20, wherein the effective plasma concentration attained by the sustained release formulation is between about 50 and about 150 ng/ml.

22. The method of claim 18, wherein said mammal is human.

23. The method of claim 21, further comprising the step of administering HAART.

24. A method for inducing latent HIV-1 expression in a mammalian cell, the method comprising administering to a mammal in need thereof a dosage amount of prostratin or a structural analog or metabolite thereof sufficient to achieve an effective plasma concentration between about 50 and about 150 ng/ml.

25. The method of claim 24, wherein said effective plasma concentration is sustained for at least 4 hours.
